SEQUENCE LISTING (28 sequences)

SEQ ID NO: 1 - DNA, 37 bp, Artificial Sequence
  L28N Forward primer
cagcccctagaagatgcaatctctcccactaccgcag

SEQ ID NO: 2 - DNA, 37 bp, Artificial Sequence
  L28N Reverse primer
ctgcggtagtgggagagattgcatcttctaggggctg

SEQ ID NO: 3 - DNA, 36 bp, Artificial Sequence
  A54N Forward primer
ggacagatatgaggaagaaaacctgagctgggggca

SEQ ID NO: 4 - DNA, 36 bp, Artificial Sequence
  A54N Reverse primer
tgcccccagctcaggttttcttcctcatatctgtcc

SEQ ID NO: 5 - DNA, 35 bp, Artificial Sequence
  P73N Forward primer
gagggaccctccaaggaatagctcctgcgctaggc

SEQ ID NO: 6 - DNA, 35 bp, Artificial Sequence
  P73N Reverse primer
gcctagcgcaggagctattccttggagggtccctc

SEQ ID NO: 7 - DNA, 29 bp, Artificial Sequence
  H97N Forward primer
gtgcttagcggccttaacaggtccgagct

SEQ ID NO: 8 - DNA, 29 bp, Artificial Sequence
  H97N Reverse primer
agctcggacctgttaaggccgctaagcac

SEQ ID NO: 9 - DNA, 26 bp, Artificial Sequence
  K154N Forward primer
ccccggtgtcggaatgcctctgtggt

SEQ ID NO: 10 - DNA, 26 bp, Artificial Sequence
  K154N Reverse primer
accacagaggcattccgacaccgggg

SEQ ID NO: 11 - DNA, 28 bp, Artificial Sequence
  A173N Forward primer
gttgcgcttggccaaccacagcgggccc

SEQ ID NO: 12 - DNA, 28 bp, Artificial Sequence
  A173N Reverse primer
gggcccgctgtggttggccaagcgcaac

SEQ ID NO: 13 - DNA, 537 bp, Artificial Sequence
  L28N DNA seqence
atgagaccttccgtctgggccgccgtggccgcaggactgtgggtcctgtgcaccgtgatcgccgcagcccctagaagatgcaatctctcccactaccgcagcctggagcccagaacactggccgctgccaaggccctgagggacagatatgaggaagaagccctgagctgggggcagcggaactgctccttccggcccaggagggaccctccaaggccaagctcctgcgctaggctcaggcacgtggctaggggaatcgccgacgctcaggctgtgcttagcggccttcacaggtccgagctgctccctggcgctggcccaattctggagctgctggccgcagcagggagggatgtggccgcctgtcttgagttggccaggccaggctctagtcggaaggtccccggggcccaaaagcggcgccataaaccccggcgggccgattcaccccggtgtcggaaggcctctgtggtctttaatctcctccggctcctgacttgggagttgcgcttggccgcccacagcgggccctgcttg

SEQ ID NO: 14 - DNA, 537 bp, Artificial Sequence
  A54N DNA seqence
atgagaccttccgtctgggccgccgtggccgcaggactgtgggtcctgtgcaccgtgatcgccgcagcccctagaagatgcctgctctcccactaccgcagcctggagcccagaacactggccgctgccaaggccctgagggacagatatgaggaagaaaacctgagctgggggcagcggaactgctccttccggcccaggagggaccctccaaggaatagctcctgcgctaggctcaggcacgtggctaggggaatcgccgacgctcaggctgtgcttagcggccttcacaggtccgagctgctccctggcgctggcccaattctggagctgctggccgcagcagggagggatgtggccgcctgtcttgagttggccaggccaggctctagtcggaaggtccccggggcccaaaagcggcgccataaaccccggcgggccgattcaccccggtgtcggaaggcctctgtggtctttaatctcctccggctcctgacttgggagttgcgcttggccgcccacagcgggccctgcttg

SEQ ID NO: 15 - DNA, 537 bp, Artificial Sequence
  P73N DNA seqence
atgagaccttccgtctgggccgccgtggccgcaggactgtgggtcctgtgcaccgtgatcgccgcagcccctagaagatgcctgctctcccactaccgcagcctggagcccagaacactggccgctgccaaggccctgagggacagatatgaggaagaagccctgagctgggggcagcggaactgctccttccggcccaggagggaccctccaaggaatagctcctgcgctaggctcaggcacgtggctaggggaatcgccgacgctcaggctgtgcttagcggccttcacaggtccgagctgctccctggcgctggcccaattctggagctgctggccgcagcagggagggatgtggccgcctgtcttgagttggccaggccaggctctagtcggaaggtccccggggcccaaaagcggcgccataaaccccggcgggccgattcaccccggtgtcggaaggcctctgtggtctttaatctcctccggctcctgacttgggagttgcgcttggccgcccacagcgggccctgcttg

SEQ ID NO: 16 - DNA, 537 bp, Artificial Sequence
  H97N DNA seqence
atgagaccttccgtctgggccgccgtggccgcaggactgtgggtcctgtgcaccgtgatcgccgcagcccctagaagatgcctgctctcccactaccgcagcctggagcccagaacactggccgctgccaaggccctgagggacagatatgaggaagaagccctgagctgggggcagcggaactgctccttccggcccaggagggaccctccaaggccaagctcctgcgctaggctcaggcacgtggctaggggaatcgccgacgctcaggctgtgcttagcggccttaacaggtccgagctgctccctggcgctggcccaattctggagctgctggccgcagcagggagggatgtggccgcctgtcttgagttggccaggccaggctctagtcggaaggtccccggggcccaaaagcggcgccataaaccccggcgggccgattcaccccggtgtcggaaggcctctgtggtctttaatctcctccggctcctgacttgggagttgcgcttggccgcccacagcgggccctgcttg

SEQ ID NO: 17 - DNA, 537 bp, Artificial Sequence
  K154N DNA seqence
atgagaccttccgtctgggccgccgtggccgcaggactgtgggtcctgtgcaccgtgatcgccgcagcccctagaagatgcctgctctcccactaccgcagcctggagcccagaacactggccgctgccaaggccctgagggacagatatgaggaagaagccctgagctgggggcagcggaactgctccttccggcccaggagggaccctccaaggccaagctcctgcgctaggctcaggcacgtggctaggggaatcgccgacgctcaggctgtgcttagcggccttcacaggtccgagctgctccctggcgctggcccaattctggagctgctggccgcagcagggagggatgtggccgcctgtcttgagttggccaggccaggctctagtcggaaggtccccggggcccaaaagcggcgccataaaccccggcgggccgattcaccccggtgtcggaatgcctctgtggtctttaatctcctccggctcctgacttgggagttgcgcttggccgcccacagcgggccctgcttg

SEQ ID NO: 18 - DNA, 537 bp, Artificial Sequence
  A173N DNA seqence
atgagaccttccgtctgggccgccgtggccgcaggactgtgggtcctgtgcaccgtgatcgccgcagcccctagaagatgcctgctctcccactaccgcagcctggagcccagaacactggccgctgccaaggccctgagggacagatatgaggaagaagccctgagctgggggcagcggaactgctccttccggcccaggagggaccctccaaggccaagctcctgcgctaggctcaggcacgtggctaggggaatcgccgacgctcaggctgtgcttagcggccttcacaggtccgagctgctccctggcgctggcccaattctggagctgctggccgcagcagggagggatgtggccgcctgtcttgagttggccaggccaggctctagtcggaaggtccccggggcccaaaagcggcgccataaaccccggcgggccgattcaccccggtgtcggaaggcctctgtggtctttaatctcctccggctcctgacttgggagttgcgcttggccaaccacagcgggccctgcttg

SEQ ID NO: 19 - DNA, 537 bp, Artificial Sequence
  L28N+P73N DNA seqence
atgagaccttccgtctgggccgccgtggccgcaggactgtgggtcctgtgcaccgtgatcgccgcagcccctagaagatgcaatctctcccactaccgcagcctggagcccagaacactggccgctgccaaggccctgagggacagatatgaggaagaagccctgagctgggggcagcggaactgctccttccggcccaggagggaccctccaaggaatagctcctgcgctaggctcaggcacgtggctaggggaatcgccgacgctcaggctgtgcttagcggccttcacaggtccgagctgctccctggcgctggcccaattctggagctgctggccgcagcagggagggatgtggccgcctgtcttgagttggccaggccaggctctagtcggaaggtccccggggcccaaaagcggcgccataaaccccggcgggccgattcaccccggtgtcggaaggcctctgtggtctttaatctcctccggctcctgacttgggagttgcgcttggccgcccacagcgggccctgcttg

SEQ ID NO: 20 - protein, 179 aa, Artificial Sequence
  WT IFN lambda 4 (Homo sapiens)
MRPSVWAAVAAGLWVLCTVIAAAPRRCLLSHYRSLEPRTLAAAKALRDRYEEEALSWGQRNCSFRPRRDPPRPSSCARLRHVARGIADAQAVLSGLHRSELLPGAGPILELLAAAGRDVAACLELARPGSSRKVPGAQKRRHKPRRADSPRCRKASVVFNLLRLLTWELRLAAHSGPCL

SEQ ID NO: 21 - protein, 158 aa, Artificial Sequence
  eIFN lambda 4 (Escherichia coli)
MAPRRCLLSHYRSLEPRTLAAAKALRDRYEEEALSWGQRNCSFRPRRDPPRPSSCARLRHVARGIADAQAVLSGLHRSELLPGAGPILELLAAAGRDVAACLELARPGSSRKVPGAQKRRHKPRRADSPRCRKASVVFNLLRLLTWELRLAAHSGPCL

SEQ ID NO: 22 - protein, 179 aa, Artificial Sequence
  L28N
MRPSVWAAVAAGLWVLCTVIAAAPRRCNLSHYRSLEPRTLAAAKALRDRYEEEALSWGQRNCSFRPRRDPPRPSSCARLRHVARGIADAQAVLSGLHRSELLPGAGPILELLAAAGRDVAACLELARPGSSRKVPGAQKRRHKPRRADSPRCRKASVVFNLLRLLTWELRLAAHSGPCL

SEQ ID NO: 23 - protein, 179 aa, Artificial Sequence
  A54N
MRPSVWAAVAAGLWVLCTVIAAAPRRCLLSHYRSLEPRTLAAAKALRDRYEEENLSWGQRNCSFRPRRDPPRNSSCARLRHVARGIADAQAVLSGLHRSELLPGAGPILELLAAAGRDVAACLELARPGSSRKVPGAQKRRHKPRRADSPRCRKASVVFNLLRLLTWELRLAAHSGPCL

SEQ ID NO: 24 - protein, 179 aa, Artificial Sequence
  P73N
MRPSVWAAVAAGLWVLCTVIAAAPRRCLLSHYRSLEPRTLAAAKALRDRYEEEALSWGQRNCSFRPRRDPPRNSSCARLRHVARGIADAQAVLSGLHRSELLPGAGPILELLAAAGRDVAACLELARPGSSRKVPGAQKRRHKPRRADSPRCRKASVVFNLLRLLTWELRLAAHSGPCL

SEQ ID NO: 25 - protein, 179 aa, Artificial Sequence
  H97N
MRPSVWAAVAAGLWVLCTVIAAAPRRCLLSHYRSLEPRTLAAAKALRDRYEEEALSWGQRNCSFRPRRDPPRPSSCARLRHVARGIADAQAVLSGLNRSELLPGAGPILELLAAAGRDVAACLELARPGSSRKVPGAQKRRHKPRRADSPRCRKASVVFNLLRLLTWELRLAAHSGPCL

SEQ ID NO: 26 - protein, 179 aa, Artificial Sequence
  K154N
MRPSVWAAVAAGLWVLCTVIAAAPRRCLLSHYRSLEPRTLAAAKALRDRYEEEALSWGQRNCSFRPRRDPPRPSSCARLRHVARGIADAQAVLSGLHRSELLPGAGPILELLAAAGRDVAACLELARPGSSRKVPGAQKRRHKPRRADSPRCRNASVVFNLLRLLTWELRLAAHSGPCL

SEQ ID NO: 27 - protein, 179 aa, Artificial Sequence
  A173N
MRPSVWAAVAAGLWVLCTVIAAAPRRCLLSHYRSLEPRTLAAAKALRDRYEEEALSWGQRNCSFRPRRDPPRPSSCARLRHVARGIADAQAVLSGLHRSELLPGAGPILELLAAAGRDVAACLELARPGSSRKVPGAQKRRHKPRRADSPRCRKASVVFNLLRLLTWELRLANHSGPCL

SEQ ID NO: 28 - protein, 179 aa, Artificial Sequence
  L28N+P73N
MRPSVWAAVAAGLWVLCTVIAAAPRRCNLSHYRSLEPRTLAAAKALRDRYEEEALSWGQRNCSFRPRRDPPRNSSCARLRHVARGIADAQAVLSGLHRSELLPGAGPILELLAAAGRDVAACLELARPGSSRKVPGAQKRRHKPRRADSPRCRKASVVFNLLRLLTWELRLAAHSGPCL

(19) United States
(12) Patent Application Publication
(10) Pub. No.: US 2023/0070752 A1
(43) Pub. Date: Mar. 9, 2023

(54) NOVEL INTERFERON LAMBDA VARIANT AND METHOD OF PRODUCING THE SAME

(71) Applicant: KOREA ADVANCED INSTITUTE OF SCIENCE AND TECHNOLOGY, Daejeon (KR)

(72) Inventors: Ho Min KIM, Daejeon (KR); Jae-Hee CHUNG, Daejeon (KR); Eui-Cheol SHIN, Gyeonggi-do (KR); Seon-Hui HONG, Gyeongsangbuk-do (KR)

(21) Appl. No.: 17/273,637

(22) PCT Filed: Aug. 19, 2020
(86) PCT No.: PCT/KR2020/011035

§ 371 Date: Oct. 17, 2022

(30) Foreign Application Priority Data
Aug. 21, 2019 (KR) 10-2019-0102220

(51) Int. Cl. C07K 14/555; A61P 3/10; A61P 31/12; A61P 35/00; A61K 38/00

(52) U.S. Cl. CPC C07K 14/555; A61P 3/10; A61P 31/12; A61P 35/00; A61K 38/00

(57) ABSTRACT

Disclosed are a novel interferon lambda variant produced through structure-based glycoengineering and a method of producing the same. The novel interferon lambda variant and the method of producing the same exhibit remarkably improved production and yield in mammalian cell lines using structural information-based glycoengineering even through conventional purification protocols, and exhibit significantly improved therapeutic properties such as stability, half-life, and fraction of functional proteins during treatment, compared to wild-type interferon lambda. In addition, the novel interferon lambda variant and the method of producing the same according to the present invention have higher antiviral activity and interferon-stimulated gene (ISG)-inducing activity than wild-type interferon lambda, and thus are useful for the prevention and treatment of immune-related diseases such as cancer and autoimmune diseases as well as various viral infections such as infection with SARS-CoV-2 (COVID-19).

### FIELD OF THE INVENTION

The present invention relates to a novel interferon lambda variant produced through structure-based glycoengineering and a method for producing the same.

### BACKGROUND OF THE INVENTION

Interferons (IFNs) are a group of cytokines that serve as the first line of defense against viruses. In addition to their protective role against viral infection, the interferon family consisting of type I, type II and type III interferons performs a wide variety of functions affecting, for example, cell growth and immune surveillance against tumor cells. All three types of interferon families activate the JAK/STAT pathway and induce interferon-stimulated gene (ISG) expression by binding to their respective receptors. The examples of the three types of interferon are: type I interferon, IFNαR1 and IFNαR2 (IFNα/β); type II interferon, IFNγR1 and IFNγR2 (IFNγ), and type III interferon, IFNλR1 and IL10Rβ (IFNλ1-4) (Nature Reviews Immunology 2005;5:375-86; Nature Reviews Immunology 2015;15:87-103; Nat. Immunol. 2015;16:802-9; The Journal of Biological Chemistry 2017;292:7295-303). In contrast to type I and type II IFN, type III IFN was only recently identified and plays not only antiviral functions but also novel immunomodulatory functions in oncology and autoimmune diseases (Drug Discovery Today 2016; 21: 167-71; Current Opinions in Immunology 2019;56:67-75). IFNλ1˜3 were identified through computer-based prediction based on genome sequencing (Nat. Immunol. 2003;4:69-77; Nat. Immunol. 2003;4:63-8), and IFNλ4 was discovered in genome-wide association studies (GWAS) in patients infected with the hepatitis C virus (HCV). The ΔG allele of the dinucleotide genetic variant (rs368234815), which is upstream of the IFNL3 locus on chromosome 19, produces functional IFNλ4, the TT allele leads to a frameshift, thereby rendering it a pseudogene (Nat. Genet. 2013;45:164-71). Interestingly, HCV patients having the ΔG allele and enhanced expression of IFNλ4 were less responsive to PEGylated-IFNα-ribavirin therapy than HCV patients having the TT allele (Nat. Commun. 2014;5:5699). However, IFNλ4 still induces the major hepatic ISG expression during chronic HCV infection and is able to drive the anti-viral response against other viruses such as the MERS-CoV in vitro (EMBO J. 2013;32:3055-65). Similar to IFNα (Roferon-A for hairy cell leukemia) and IFNβ (Avonex for multiple sclerosis), the phase 2 clinical trial of PEGylated IFNλ1 regarding hepatitis D virus (HDV) infection highlights the pharmaceutical potential of the IFNλ family.

Meanwhile, in conventional mammalian cells, transient expression of wild-type IFNλ4 is insufficient to produce an effective amount of recombinant IFNλ4. There is an opinion that weak signal peptides in IFNλ4 may cause impaired secretion of IFNλ4 and appropriate glycosylation of IFNλ4 may be required for secretion (Nat. Genet. 2013;45:164-71). Recombinant IFNλ4 can be purified from a bacterial expression system by refolding the inclusion body (EMBO J. 2013;32:3055-65), but the refolding method causes a number of problems, such as complexity of the purification step, lack of glycosylation, and endotoxin contamination, and further, the lack of glycosylation may affect the efficacy of IFNλ4.

Recently, glyco-moieties may affect various protein properties, such as improvement of solubility, stability, in-vivo activity, plasma half-life and productivity. Thus, glycoengineering techniques for introducing new glycosylation sites or altering the glycan composition of CHO cells have come to be widely used to improve therapeutic proteins. For example, half-life and productivity are improved through glycoengineering of hIFNβ-1a and hIFNα (PLoS One 2014;9:e96967; Biochimie 2008;90:437-49). Moreover, the addition of a single N-glycosylation site may increase the secretion of lipase, cutinase, llama VHH antibody and macrophage inhibitory cytokine 1 (Applied and Environmental Microbiology 2000;66:4940-4; Biotechnology Progress 2009;25:1468-75).

Against this background, the present inventors produced various IFNλ4 variants through mutagenesis in order to introduce new potential N-glycosylation sites based on the model structure of the IL10Rβ-IFNλ4-IFNλR1 complex to improve the expression level and therapeutic properties of IFNλ4 through glycoengineering of IFNλ4. In particular, the present inventors found that L28N, P73N, and L28N+P73N variants exhibited improved productivity, and in particular, P73N showed a new glycosylation site. In addition, the present inventors found that the HEK293-expressed IFNλ4 variant of the present invention exhibits remarkably stronger IFNλ4-mediated signaling and antiviral activity than IFNλ4 derived from E. coli while maintaining binding affinity for IL10Rβ and IFNλR1 receptors. Based on these findings, the present invention has been completed.

The information disclosed in this Background section is provided only for better understanding of the background of the present invention, and therefore it may not comprise information that forms the prior art that is already obvious to those skilled in the art.

### SUMMARY OF THE INVENTION

Therefore, the present invention has been made in view of the above problems, and it is one object of the present invention to provide a novel interferon lambda variant that has significantly improved expression yield, higher stability, a longer half-life, better antiviral activity and better interferon-stimulating gene induction activity than an interferon lambda protein through structure-based glycoengineering.

It is another object of the present invention to provide the use of the novel interferon lambda variant for immunomodulation.

It is another object of the present invention to provide the use of the novel interferon lambda variant for the prevention and treatment of viral infections.

It is another object of the present invention to provide the use of the novel interferon lambda variant for the prevention and treatment of cancer, tumors, organ transplant rejection (transplant rejection), chronic renal failure, cirrhosis, diabetes or hyperglycemia.

It is another object of the present invention to provide a method for producing the interferon lambda variant through structure-based glycoengineering.

In accordance with one aspect of the present invention, the above and other objects can be accomplished by the provision of an interferon lambda (IFNλ) variant comprising a mutation at at least one site that satisfies at least one of the following criteria:

(i) a site is positioned outside an interferon lambda receptor-binding region;

(ii) a varied amino acid residue is exposed to the surface of interferon lambda; and

(iii) a consensus sequence enabling glycosylation is achieved through a single point mutation,

wherein the consensus sequence enabling glycosylation is N-X-(S or T), in which X is an amino acid other than proline.

In accordance with another aspect of the present invention, there is provided a gene encoding the interferon lambda variant.

In accordance with another aspect of the present invention, there is provided a recombinant vector comprising the gene.

In accordance with another aspect of the present invention, there is provided a recombinant cell introduced with the gene or the recombinant vector.

In accordance with another aspect of the present invention, there is provided a composition for immunomodulation comprising the interferon lambda variant.

In accordance with another aspect of the present invention, there is provided the use of the interferon lambda variant for immunomodulation.

In accordance with another aspect of the present invention, there is provided a method for immunomodulation comprising treating or administering the interferon lambda variants or the composition for immunomodulation.

In accordance with another aspect of the present invention, there is provided a composition comprising the interferon lambda variant for preventing and treating viral infections.

In accordance with another aspect of the present invention, there is provided a method for preventing and treating viral infections comprising administering the interferon lambda variant or the composition comprising the same according to the present invention to a subject.

In accordance with another aspect of the present invention, there is provided the use of the interferon lambda variant for the prevention and treatment of viral infections.

In accordance with another aspect of the present invention, there is provided a composition for preventing and treating immune diseases comprising the interferon lambda variant.

In accordance with another aspect of the present invention, there is provided a method for preventing and treating immune diseases comprising administering the interferon lambda variant or the composition comprising the same to a subject.

In accordance with another aspect of the present invention, there is provided the use of the interferon lambda variant for the prevention and treatment of immune diseases.

In accordance with another aspect of the present invention, there is provided a pharmaceutical composition for preventing and treating cancer, tumors, organ transplant rejection (transplant rejection), chronic renal failure, cirrhosis, diabetes or hyperglycemia comprising the novel interferon lambda variant.

In accordance with another aspect of the present invention, there is provided the use of the novel interferon lambda variant for the prevention and treatment of cancer, tumors, organ transplant rejection (transplant rejection), chronic renal failure, cirrhosis, diabetes or hyperglycemia.

In accordance with another aspect of the present invention, there is provided a method for preventing and treating cancer, tumors, organ transplant rejection (transplant rejection), chronic renal failure, cirrhosis, diabetes or hyperglycemia, comprising administering the interferon lambda variant or the composition comprising the same according to the present invention to a subject.

In accordance with another aspect of the present invention, there is provided a method of producing an interferon lambda (IFNλ) variant, wherein the method comprising:

expressing an interferon lambda (IFNλ) variant comprising a mutation at at least one site of interferon lambda that satisfies at least one of the following criteria:

(i) a site is positioned outside an interferon lambda receptor-binding region;

(ii) the varied amino acid residue is exposed to the surface of interferon lambda; and

(iii) a consensus sequence enabling glycosylation is achieved through a single point mutation,

wherein the consensus sequence enabling glycosylation is N-X-(S or T), in which X is an amino acid other than proline; and

collecting the interferon lambda (IFNλ) variant.

### BRIEF DESCRIPTION OF THE DRAWINGS

The above and other objects, features and other advantages of the present invention will be more clearly understood from the following detailed description taken in conjunction with the accompanying drawings, in which:

FIG. 1 relates to the design of an IFNλ4 variant.

FIG. 1A shows a model structure (right) of IL10Rβ-IFNλ4-IFNλR1 constructed from IL10Rβ-IFNλ3-IFNλR1 (PDB code: 5T5W, left), wherein potential N-glycosylation mutagenic sites (L28, A54, P73, H97, K154, A173) are represented in orange in the structure, and the endogenous N-glycosylation site N61 is represented in blue in the structure.

FIG. 1B shows alignment of the sequence of the IFNλ4 protein, wherein amino acids important for binding to IFNλR1 and IL10Rβ are highlighted in green and cyan, respectively, potential N-glycosylation mutagenic sites are represented by orange boxes (M1-M6), the endogenous N-glycosylation site N61 is represented by a blue box (M0), conservation of the sequence is represented in the order of red, blue and black in descending order of conservation rate and the *IFNλ3 sequence was obtained from the crystal structure of the IL10Rβ-IFNλ3-IFNλR1 complex having an affinity-enhancing mutation in IFNλ3 to stabilize the interaction with IL10Rβ.

FIG. 1C shows the binding mode of IFNλ3 and IFNλ4 towards IL10Rβ. The hydrogen bond network between IFNλ3 and IL10Rβ is shown and similar interactions between IFNλ4 and IL10Rβ are mapped based on the model structure of IL10Rβ-IFNλ4-IFNλR1 (left).

FIG. 2 relates to the production of the IFNλ4 variant.

FIG. 2A shows the result of the expression test of the IFNλ4 variant Expression level of IFNλ4 wild-type and variants comprising C-terminal 6×-histidine tags were monitored by Western blot with anti-his antibody. M1 (L28N), M3 (P73N), and M7 (L28N+P73N) showed enhanced expression and were selected for larger scale expression.

FIG. 2B shows the results of Coomassie blue staining of M1 (L28N), M3 (P73N) and M7 (L28N+P73N) purified under reducing and non-reducing condition. The proteins were purified by affinity chromatography with IgG sepharose followed by thrombin digestion and gel filtration chromatography.

FIG. 2C shows gel filtration Gel filtration chromatogram of M1, M3, M7, and standard proteins. Each gel filtration peak corresponds to standard proteins: thyroglobulin (670 kDa), γ-globulin (158 kDa), ovalbumin (44 kDa), myoglobin (17 kDa), and vitamin B12 (1.35 kDa).

FIG. 3 shows the result of confirming the N-glycosylation of the IFNλ4 variant.

FIG. 3A shows SDS-PAGE analysis with and without PNGase-F treatment of IFNλ4 variants, M1, M3, and M7.

FIG. 3B is a Schematic diagram of IFNλ4 variants, M1, M3, and M7, marked with the confirmed position of N-glycosylation by mass spectrometry.

FIG. 3C is a collision-induced dissociation (CID) tandem MS spectrum of precursor ions at m/z 813.95 [M+3H]³⁺ corresponding to Hex5HexNAc4Fuc1NeuAc1 (NCS) having a peptide backbone on N61 from the IFNλ4 variant.

FIG. 3D is a collision-induced dissociation (CID) tandem MS spectra of precursor ion at m/z 745.93 [M+3H]3+ corresponding to Hex5HexNAc4Fuc1 with peptide back bone (NSSC) on P73N from IFNλ4 variants (M3 and M7). Mutated L28Ns in M1 and M7 were not glycosylated.

FIG. 4 shows the binding kinetics of the IFNλ4 variant to IFNλR1 or IL10Rβ.

FIG. 4A shows Binding curves of IFNλR1 and IL10Rβ toward IFNλ4 variants (M1, M3, M7) and eIFNλ4 at the indicated concentrations of IFNλR1 and IL10Rβ (500, 1000, 2000 nM). Sensorgrams were obtained from BLItz instrument. Data points are shown in grey and the corresponding fits are shown in red (IFNλR1) and blue (IL10Rβ). KD values were calculated from 1:1 global fitting.

FIG. 4B shows Binding kinetics of IFNλR1 and IL10Rβ to immobilized IFNλ4 variants (M1, M3, M7) and eIFNλ4. Goodness of fit was assessed by evaluating the x2 and R2 values generated for all fitting analyses.

FIG. 5 shows a result of confirming the biological activity of the IFNλ4 variants.

FIG. 5A shows the effect of activating the JAK-STAT pathway by IFNλ4 variants via the IFNλ receptor. Huh-7.5 cells were treated with IFNλs or IFNλ4 variants (10 nM) for 30 min. The expression of IFNλR1 was suppressed by IFNλR1-specific siRNA (siIFNλR1) to show changes in the phosphorylation level of STAT1 (pSTAT1) triggered by IFNλ4 variants (eIFNλ4, M1, M3, and M7 IFNλ4 variants).

FIG. 5B shows the effect of inducing the interferon-stimulated gene 15 (ISG15) expression by IFNλ4 variants in Huh-7.5 cells. Huh-7.5 cells were treated with 10 nM IFNλs for 10 hours.

FIG. 5C shows the effect of inhibiting the HCV replication by IFNλ4 variants. HCVcc-infected Huh-7.5 cells were treated with the indicated concentration of IFNλs for 48 hours.

FIG. 5D shows the production of U-ISGF3 by the prolonged treatment of IFNλ4 variants. Huh-7.5 cells were treated with 10 nM IFNα, IFNβ, IFNγ, or IFNλs for 72 hr. Extended exposure to type III interferons induced the expression of U-ISGF3, which was composed of IRF9, unphosphorylated STAT1, and unphosphorylated STAT2. Similar responses by IFNλ4 variants were monitored.

FIG. 5E shows the Up-regulation of Mx1 after prolonged treatment with IFNλs (10 nM). Mx1 is preferentially expressed by U-ISGF3 after prolonged treatment with type III interferons.

FIG. 5F shows the immunoblot result of USP18 and SOCS1 48 hours after treatment of Huh-7.5 cells with IFNλ at a final concentration of 10 nM, wherein the intensity of β-actin versus USP18 (USP18/β-actin) is plotted as a bar graph at the bottom.

FIG. 5G shows the Induction of SOCS1 expression by 10 nM IFNλs treatment in Huh-7.5 cells. Relative expression was determined at 8- and 24-hour post-treatment by real-time quantitative PCR.

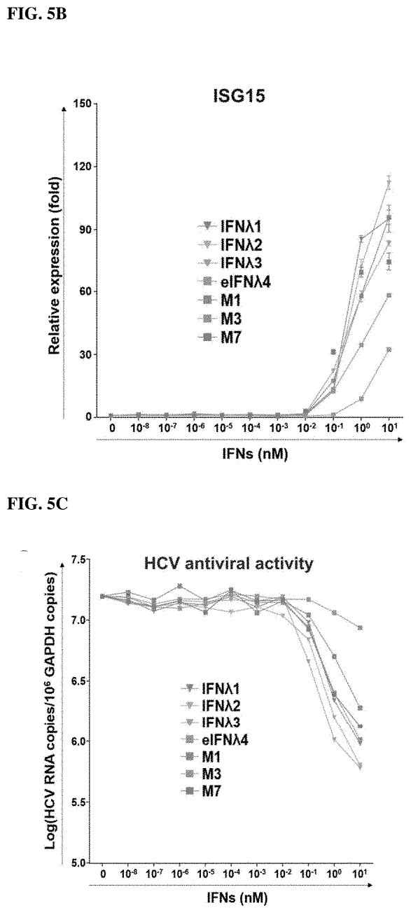

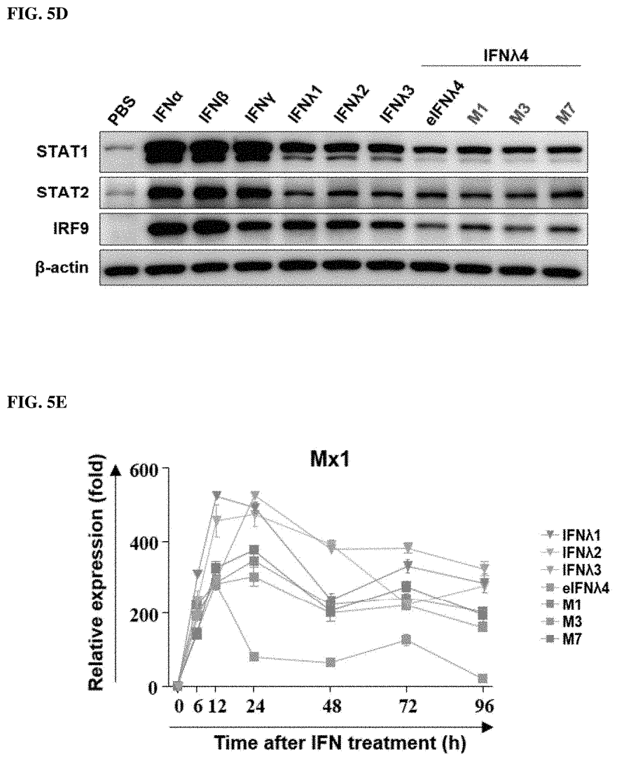

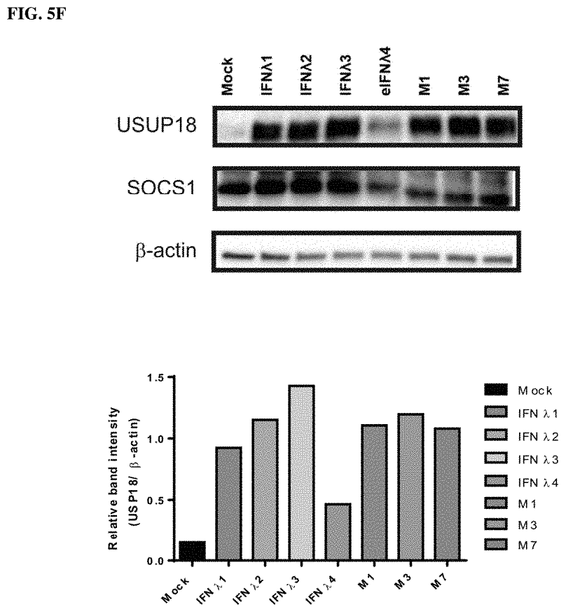

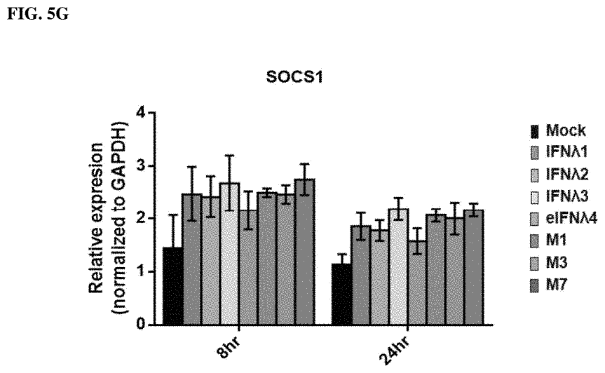

In FIGS. 5A, 5D and 5F, immunoblots are representative of three independent experiments with similar results.

In FIGS. 5B, 5C, 5E and 5G, all analyses were done with triplicates and the graphs are representative of three independent experiments with similar results.

### DETAILED DESCRIPTION OF THE INVENTION

Unless defined otherwise, all technical and scientific terms used herein have the same meanings as appreciated by those skilled in the field to which the present invention pertains. In general, the nomenclature used herein is well-known in the art and is ordinarily used.

To date, studies of interferon lambda and the clinical therapeutic use thereof have faced obstacles due to the low expression yield thereof. In particular, the role of the interferon lambda 4 (IFNλ4) in hepatitis C virus (HCV) infection has recently been known and studied, but the clinical potential therefor is considerably limited due to low expression in vitro. In an attempt to solve this problem, a conventional method for purifying bacteria-derived recombinant IFNλ4 through refolding (EMBO J. 2013;32:3055-65) causes a lot of problems such as complexity of the purification step, lack of glycosylation and endotoxin contamination.

In an embodiment of the present invention, in order to solve this problem, the structure of IL10Rβ-IFNλ4-IFNλR1 was modeled based on the IL10Rβ-IFNλ3-IFNλR1 crystal structure. Based on this structure, the following mutation site criteria for the interferon lambda 4 variant were designed, and the mutation site was screened.

In another embodiment of the present invention, a recombinant interferon lambda 4 variant was produced based on the screened mutation sites, and some of the produced variants exhibited much higher expression and production ability as well as improved therapeutic properties and biological activity than wild-type interferon lambda 4.

The variant of the present invention was designed based on the crystal structure of interferon lambda binding to the receptor and thus is not limited to interferon lambda 4, as is used in one embodiment, and can be extended to type III interferon (interferon lambda), in which a conserved sequence is maintained.

Compared to the wild-type, the novel interferon lambda variant produced through the structure-based design of the present invention has remarkably high expression rate, and excellent therapeutic properties such as increased stability and half-life through charge balance, and furthermore significantly improves expression of immune-related genes and induction of an immune response.

Accordingly, in one aspect, the present invention is directed to an interferon lambda (IFNλ) variant comprising a mutation at at least one site that satisfies at least one of the following criteria:

(i) a site is positioned outside an interferon lambda receptor-binding region;

(ii) the varied amino acid residue is exposed to the surface of interferon lambda; and

(iii) a consensus sequence enabling glycosylation is achieved through a single point mutation,

wherein the consensus sequence enabling glycosylation is N-X-(S or T), in which X is an amino acid other than proline.

As used herein, the term “interferon lambda (IFNλ)” refers to a type III interferon group and is represented by interferon lambdas 1 to 4. An interferon lambda protein is involved in an immune response against viral infection and is known to have antiviral and antiproliferative effects through the JAK-STAT pathway.

As used herein, the term “mutation” refers to deletion, insertion or substitution of nucleotide or amino acid residues occurring by chemical means, enzymatic means or various other known means in a reference sequence (e.g., a polynucleotide sequence encoding a wild-type interferon or an amino acid sequence of a wild-type interferon). In one embodiment of the present invention, the mutation causes mutagenesis in the gene sequence encoding the wild-type interferon lambda 4 protein, so that the amino acid encoded by the corresponding mutation portion is substituted asparagine (N), but the invention is not limited thereto.

As used herein, the term “interferon lambda variants” means an interferon lambda protein that has an amino acid sequence different from that of a wild-type interferon antibody, or is characterized by addition, deletion and substitution of additional components such as carbohydrates, amino acids and lipids, or has different secondary and tertiary structures. In the present invention, the interferon lambda variant may be an interferon lambda protein further comprising another amino acid sequence and/or glycosylation site, but is not limited thereto.

In one embodiment of the present invention, a variant was produced based on the sequence of wild-type interferon lambda 4 (SEQ ID NO: 20, NCBI Accession Number: AFQ38559.1), which is type III interferon, and in order to increase the expression rate without negatively affecting biological activity in numerous variants of interferon lambda 4, the mutation sites were screened based on three criteria for glycoengineering. All forms of type III interferon (interferon lambda) are known to form a complex with two types of interferon lambda receptors, IL10Rβ and IFNLR1, and to be involved in the immune response through signaling (The Journal of General Virology. 86 (Pt 6): 1589-96.). Therefore, the three criteria were designed through glycoengineering based on IL10Rβ-IFNλ4-IFNλR1 modeled based on the crystal structure of IL10Rβ-IFN3-IFNλR1, which is an interferon-lambda/receptor complex. the mutation sites for new N-glycosylation were screened based on three criteria: i) the mutation site should be outside the receptor binding region to minimize the change in the receptor-ligand binding and signal activation, and ii) should be exposed to the solvent to allow access to oligosaccharyltransferase (OST), which catalyzes the initial transfer of glycan from the lipid-linked oligosaccharide onto the substrate asparagine, and iii) the consensus sequence (NXS/T, X=any amino acid except proline) should be achieved by single point mutation at the mutation site to minimize the structural distortion caused by the mutation.

Accordingly, in the present invention, the interferon lambda receptor in the criterion (i) may be IL10Rβ and/or IFNλR1.

In the present invention, the criterion (ii) is that the varied amino acid residue is exposed on the surface of the three-dimensional structure of interferon lambda.

In the present invention, glycosylation does not always occur at the mutation site. In one embodiment of the present invention, a variant was produced based on glycoengineering, and the M3 (P73N) and M7 (L28N+P73N) IFNλ4 variants have N-glycosylation occurring in another mutation site P73N in addition to the existing N-glycosylation site. However, in M1 (L28N) among the variants showing an increase in the expression rate, N-glycosylation did not occur at the mutation site.

In the present invention, at least one site of interferon lambda may be glycosylated due to the mutation, and preferably, the mutation site may be glycosylated.

As used herein, the term “glycosylation” indicates the most common form of modification of protein such as serine or asparagine, and refers to a process in which carbohydrate glycan binds to an amino acid residue, for example, oxygen in serine or to nitrogen in asparagine. The glycosylation may affect various properties, such as secondary and tertiary protein structures, intercellular signaling, biological activity, and stability.

In the present invention, the glycosylation may be N-glycosylation (N-(linked) glycosylation), O-glycosylation (O-(linked) glycosylation), phosphoserine glycosylation, C-mannosylation, or the like, preferably N-glycosylation or O-glycosylation, and in one embodiment of the present invention, N-glycosylation is induced, but the invention is not limited thereto.

In the present invention, sugars such as mannose, fucose, galactose and GlcNAc may be added to the amino acid residue through the glycosylation, but the invention is not limited thereto.

In the present invention, the varied amino acid is glycosylated. Most preferably, glycosylation occurs when the sugar is bonded to the position P73.

In the present invention, the interferon lambda variant exhibits improved binding affinity for IL10Rβ, and when the interferon lambda is interferon lambda 4, it could have a KD value for IL10Rβ of 40 to 50 nM.

In the present invention, the interferon lambda variant exhibits binding affinity to IFNλR1 similar to that of wild-type interferon lambda or improved compared thereto, and when the interferon lambda is interferon lambda 4, it has a KD value for IFNλR1 of 10 to 25 nM.

In the present invention, the interferon lambda variant has reduced net charge compared to the wild-type interferon lambda by acidic N-glycosylation or the like, and has improved stability through the balance of charges.

In the present invention, the interferon lambda variant may have an increased half-life in vivo compared to wild-type interferon lambda. In the present invention, the interferon lambda variant may particularly represent high fraction of a protein that exhibits functional activity during a continuous treatment process.

In the present invention, the interferon lambda variant may exhibit antiviral activity and activity of inducing an interferon-stimulated gene (ISG).

In an embodiment of the present invention, the M1 variant is found to exhibit remarkably excellent expression and production yield even though no additional glycosylation occurred at the mutation site, it may be related with L28 acts as a hydrophobic aggregation nucleus to thereby interact with a hydrophobic residue such as L29 or Y32 (Proc. Natl. Acad. Sci. USA 2009;106:11937-42). Accordingly, in the present invention, the interferon lambda variant may have reduced hydrophobic interaction between interferon lambda molecules, compared to wild-type interferon lambda.

In the present invention, the interferon lambda is preferably interferon lambda 4 (IFNλ4), as can be seen from Examples, but the variant of the present invention is designed based on the crystal structure modeled based on the structure of a complex with interferon lambda 3 receptors (IL10Rβ and IFNλR1), and can be extended not only to interferon lambda 3 (IFNλ3) but also to all type III interferons (such as IFNλ1, IFNλ2 and IFNλ3) that maintain a conserved sequence.

As used herein, the term “reference sequence” refers to an amino acid sequence of a lambda protein to be varied in the present invention, or a nucleic acid sequence encoding the variant of the present invention. A conserved sequence capable of exhibiting the biological activity of interferon lambda can be maintained between the reference sequences.

In the present invention, the reference sequence is preferably a wild-type interferon lambda sequence, but may be a homologous protein or other variant thereof that shares a conserved sequence, and when the interferon lambda is interferon lambda 4, the reference sequence is preferably the sequence of SEQ ID NO: 20, as in the embodiment of the present invention, but is not limited thereto.

In the present invention, when the interferon lambda is IFNλ4, the mutation site may be selected from L28, A54, P73, H97, K154 and A173 of SEQ ID NO: 20, and when the interferon lambda is another interferon lambda protein (e.g., IFNλ1 to IFNλ3), the mutation site may be selected from amino acids corresponding to L28, A54, P73, H97, K154 and A173 of SEQ ID NO: 20.

As used herein, the term “corresponding amino acid” refers to an amino acid corresponding to the amino acid at the position of SEQ ID NO: 20 when the amino acid sequence of another interferon lambda protein (e.g., IFNλ1 to IFNλ4) is aligned with IFNλλ4 (SEQ ID NO: 20).

In the present invention, the mutation may be substitution of at least one amino acid in the amino acid sequence of interferon lambda, and preferably, for glycosylation, the amino acid may be substituted with asparagine (N) or serine (S). In the present invention, the mutation may be substitution of the amino acid at at least one position of L28, A54, P73, H97, K154 and A173 in SEQ ID NO: 20 with asparagine. In the present invention, most preferably, the mutation may be substitution of the amino acid at at least one position of L28N and P73N in SEQ ID NO: 20 with asparagine.

In the present invention, the interferon lambda variant may comprise any one amino acid sequence of SEQ ID NO: 22 to SEQ ID NO: 28, most preferably SEQ ID NO: 22, 24 or 28.

In another aspect, the present invention is directed to a gene encoding the interferon lambda variant.

In the present invention, the interferon lambda variant may share the same characteristics and embodiments as described above.

In the present invention, the gene may comprise nucleic acid sequences represented by SEQ ID NOS: 13 to 19, preferably SEQ ID NOS: 13, 15 and 19. In the present invention, the gene encoding the interferon lambda variant may further comprise protein A or a tag sequence such as a 6×-His tag at the end for purification.

In another aspect, the present invention is directed to a recombinant vector comprising a gene encoding the interferon lambda variant.

Any vector known in the art can be appropriately selected and used as the recombinant vector by those skilled in the art without limitation, so long as it is capable of inducing the expression of a protein encoded by the introduced gene. For example, when E. coli is used as a host, vectors comprising T7 series (T7A1, T7A2, T7A3, etc.), lac, lacUV5, temperature-dependent (λpL, λpR), phoA, phoB, rmB, tac, trc, trp or 1PL promoters may be used. When yeast is used as a host, a vector comprising the ADH1, AOX1, GAL1, GAL10, PGK or TDH3 promoter may be used, and when Bacillus is used as a host, a vector comprising the P2 promoter may be used. These are provided only as representative embodiments and, in addition to the vectors comprising the promoters, any vector can be appropriately selected from various vectors known in the art by those skilled in the art without any limitation so long as it is suitable for a host as a vector comprising a promoter for inducing the expression of the interferon lambda variant according to the present invention.

In another aspect, the present invention is directed to a recombinant cell introduced with the gene or the recombinant vector.

In the present invention, the recombinant cell refers to a cell for expression introduced with a gene or a recombinant vector to produce a protein or the like. The recombinant cell may be used without limitation as long as it is a cell capable of expressing glycosylated interferon lambda, and may preferably be a eukaryotic cell, more preferably yeast, an insect cell, or an animal cell, and most preferably an animal cell. For example, a CHO cell line or a HEK cell line mainly used for expression of recombinant proteins may be used, and in one embodiment of the present invention, an Expi293 cell line, which is a HEK cell line, was used, but the invention is not limited thereto. In the present specification, the term “recombinant cell” is used interchangeably with “host cell” or “recombinant host cell” having the same sense.

As used herein, the term “vector” means a DNA product comprising a DNA sequence operably linked to a suitable regulatory sequence capable of expressing the DNA in a suitable host. Vectors may be plasmids, phage particles or simply potential genomic inserts. When transformed into a suitable host, vectors may be replicated or perform functions independent of the host genomes, or some thereof may be integrated with the genomes. A plasmid is currently the most commonly used form of vector, and thus the terms “plasmid” and “vector” are often used interchangeably. However, the present invention encompasses other forms of vectors that are known in the art or have the same functions as those known in the art. Protein expression vectors used in E. coli comprise: the pET family vectors from Novagen, Inc (USA); the pBAD family vectors from Invitrogen Corp. (USA); PHCE or pCOLD vectors from Takara Bio Inc. (Japan); and pACE family vectors from GenoFocus Inc. (South Korea). In Bacillus subtilis, a gene of interest can be inserted into a specific part of the genome to realize protein expression, or a pHT-family vector of MoBiTech (Germany) can be used. Even in fungi and yeast, protein expression is possible using genome insertion or self-replicating vectors. A plant protein expression vector using a T-DNA system such as Agrobacterium tumefaciens or Agrobacterium rhizogenes can be used. Typical expression vectors for expression in mammalian cell cultures are based on, for example, pRK5 (EP 307,247), pSV16B (WO 91/08291), and pVL1392 (Pharmingen).

As used herein, the term “expression control sequence” means a DNA sequence essential for the expression of a coding sequence operably linked to a particular host organism. Such a control sequence comprises promoters for conducting transcription, operator sequences for controlling such transcription, sequences for encoding suitable mRNA ribosome-binding sites, and sequences for controlling the termination of transcription and translation. For example, control sequences suitable for prokaryotes comprise promoters, optionally operator sequences, and ribosome-binding sites. Control sequences suitable for eukaryotic cells comprise promoters, polyadenylation signals, and enhancers. The factor that has the greatest impact on the expression level of a gene in a plasmid is the promoter. SRα promoters, cytomegalovirus-derived promoters and the like are preferably used as promoters for high expression.

Any of a wide variety of expression control sequences may be used for the vector in order to express the DNA sequences of the present invention. Useful expression control sequences comprise, for example, early and late promoters of SV40 or adenovirus, the lac system, the trp system, the TAC or TRC system, T3 and T7 promoters, the major operator and promoter regions of phage lambda, control regions of fd code proteins, promoters of 3-phosphoglycerate kinase or other glycol lyases, promoters of the phosphatase, such as Pho5, promoters of yeast alpha-mating systems, and other sequences having configurations and induction activity known to control gene expression of prokaryotic or eukaryotic cells or viruses and various combinations thereof. The T7 RNA polymerase promoter Φ10 may be useful for expressing proteins in E. coli.

When a nucleic acid sequence is aligned with another nucleic acid sequence based on a functional relationship, it is “operably linked” thereto. This may be gene(s) and control sequence(s) linked in such a way so as to enable gene expression when a suitable molecule (e.g., a transcriptional activator protein) is linked to the control sequence(s). For example, DNA for a pre-sequence or secretory leader is operably linked to DNA for a polypeptide when expressed as a pre-protein involved in the secretion of the polypeptide; a promoter or enhancer is operably linked to a coding sequence when it affects the transcription of the sequence; or a ribosome-binding site is operably linked to a coding sequence when it affects the transcription of the sequence; or the ribosome-binding site is operably linked to a coding sequence when positioned to facilitate translation. Generally, the term “operably linked” means that the linked DNA sequence is in contact therewith, and that a secretory leader is in contact therewith and is present in the reading frame. However, the enhancer need not be in contact therewith. The linkage of these sequences is carried out by ligation (linkage) at convenient restriction enzyme sites. When no such site exists, a synthetic oligonucleotide adapter or a linker according to a conventional method is used.

As used herein, the term “expression vector” commonly refers to a recombinant carrier into which a fragment of heterologous DNA is inserted, and generally means a fragment of double-stranded DNA. Herein, “heterologous DNA” means xenogenous DNA that is not naturally found in the host cell. Once an expression vector is present in a host cell, it can replicate independently of the host chromosomal DNA, and several copies of the vector and inserted (heterologous) DNA thereof can be produced.

As is well known in the art, in order to increase the expression level of a transfected gene in a recombinant cell, the gene should be operably linked to a transcriptional or translational expression control sequence that functions in the selected expression host. Preferably, the expression control sequence and the corresponding gene are comprised in a single expression vector comprising both a bacterial selection marker and a replication origin. When the expression host is a eukaryotic cell, the expression vector should further comprise a useful expression marker in the eukaryotic expression host.

In the present invention, as the host cells for expressing the recombinant protein, prokaryotic cells such as Escherichia coli and Bacillus subtilis, which enable cell culture at a high concentration within a short time, can be easily genetically manipulated, and of which the genetic and physiological characteristics are well known, have been widely used. However, in order to solve problems such as post-translational modification of proteins, secretion process and three-dimensional structure of the active form, and the active state of the protein, single-celled eukaryotic yeasts (such as Pichia pastoris, Saccharomyces cerevisiae, and Hansenula polymorpha), filamentous fungi, insect cells, plant cells, and cells of higher organisms such as mammals have been recently used as host cells for the production of recombinant proteins. Thus, the use of other host cells as well as Bacillus bacillus described in the examples will be readily evident to those of ordinary skill in the art. For example, a CHO cell line, a HEK cell line or the like may be used as a host cell for expression, but is not limited thereto.

A wide variety of expression host/vector combinations can be used to express the interferon lambda variants of the present invention. Suitable expression vectors for eukaryotic hosts comprise, for example, expression control sequences derived from SV40, cow papillomavirus, adenovirus, adeno-associated virus, cytomegalovirus and retrovirus. Expression vectors that can be used for bacterial hosts comprise bacterial plasmids that may be exemplified by those obtained from E. coli, such as pBlueScript, pGEX2T, pUC vectors, col E1, pCR1, pBR322, pMB9 and derivatives thereof, plasmids having a wide host range such as RP4, phage DNA that may be exemplified by a wide variety of phage lambda derivatives such as λgt10, λgt11 and NM989, and other DNA phages such as M13 and filamentous single-stranded DNA phages. Expression vectors useful for yeast cells comprise 2 μ plasmids and derivatives thereof. A vector useful for insect cells is pVL 941.

The recombinant vector may be introduced into a host cell through a method such as transformation or transfection. As used herein, the term “transformation” means introducing DNA into a host and making the DNA replicable using an extrachromosomal factor or chromosomal integration. As used herein, the term “transfection” means that an expression vector is accommodated by the host cell, regardless of whether or not any coding sequence is actually expressed.

It should be understood that not all vectors and expression control sequences function identically in expressing the DNA sequences of the present invention. Similarly, not all hosts function identically in the same expression system. However, those skilled in the art will be able to make appropriate selection from a variety of vectors, expression control sequences and hosts without excessive burden of experimentation without departing from the scope of the present invention. For example, selection of a vector should be carried out in consideration of the host, because the vector should be replicated therein. The number of replications of the vector, the ability to control the number of replications, and the expression of other proteins encoded by the corresponding vector, such as the expression of antibiotic markers, should also be considered. In selecting the expression control sequence, a number of factors should be considered. For example, the relative strength of the sequence, controllability, and compatibility with the DNA sequences of the present invention should be considered, particularly in relation to possible secondary structures. A single-celled host may be selected in consideration of factors such as the selected vector, the toxicity of the product encoded by the DNA sequence of the present invention, secretion characteristics, the ability to accurately fold proteins, culture and fermentation factors, and ease of purification of the product encoded by the DNA sequence according to the present invention from the host. Within the scope of these factors, those skilled in the art can select various vector/expression control sequences/host combinations capable of expressing the DNA sequences of the present invention in fermentation or large animal cultures. As a screening method for cloning cDNA of proteins through expression cloning, a binding method, a panning method, a film emulsion method or the like can be applied.

The gene and recombinant vector may be introduced into host cells through various methods known in the art. The gene encoding the interferon lambda variant of the present invention may be directly introduced into the genome of a host cell and present as a factor on a chromosome. It will be apparent to those skilled in the art to which the present invention pertains that even if the gene is inserted into the genomic chromosome of the host cell, it will have the same effect as when the recombinant vector is introduced into the host cell.

In the present invention, specific amino acid sequences and nucleotide sequences have been described, but it will be apparent to those skilled in the art that the amino acid sequences substantially identical to the enzymes to be implemented in the present invention and the nucleotide sequences encoding the same fall within the scope of the present invention. “Substantially identical” comprises the case in which the homology of an amino acid or a nucleotide sequence is very high, and means a protein that shares structural features regardless of sequence homology or has the same function as used in the present invention. A protein from which a sequence other than the sequence constituting the subject matter of the present invention is partially deleted or a fragment of a nucleotide sequence encoding the same may fall within the scope of the present invention. Therefore, the present invention comprises all amino acid or nucleotide sequences having the same function as used in the present invention regardless of the length of the fragment.

In one embodiment of the present invention, it was found that the produced novel interferon lambda 4 variant exhibits antiviral activity remarkably superior to that of wild-type interferon lambda 4 and induces expression of a similar or upregulated IFN signaling factor, which means that the interferon lambda 4 variant exhibits remarkably superior biological activity to eIFNλ4, which is wild-type IFNλ4.

In another aspect, the present invention is directed to the use of the interferon lambda variant for immunomodulation.

In another aspect, the present invention is directed to a composition for immunomodulation comprising the interferon lambda variant.

In another aspect, the present invention is directed to a method for immunomodulation comprising treating or administering the interferon lambda variant or the composition for immunomodulation comprising the same.

As herein used, the term “immunomodulation” means overcoming an immune imbalance in the blood, while maintaining immune homeostasis. Maintenance of immune homeostasis refers to a state of a balance between immune tolerance to suppress immunity and immune response to increase immunity. Maintenance of such a state is an essential part of the treatment of diseases, such as cancer and autoimmune diseases, pertaining to immunomodulatory abnormalities, as the cause or symptoms of illness. In the present invention, the immunomodulation is preferably immunity enhancement, and in particular, most preferably regulation of the immune response through the JAK-STAT pathway, in which interferon lambda is involved.

In the present invention, the composition for immunomodulation can significantly up-regulate the expression of interferon-stimulated gene (ISG).

The composition for immunomodulation can be used as a pharmaceutical composition or a health functional food for controlling immune activity, and preventing, ameliorating or treating various infectious diseases involving viruses and bacteria and immune-related diseases, and the amount and form of use can be adjusted depending on the purpose.

As herein used, the term “subject” refers to a subject to which the interferon lambda variant or the composition for various applications comprising the same according to the present invention is administered, and the subject comprises all of cells and tissues as well as various plants, animals and the like, preferably humans.

In another aspect, the present invention is directed to the use of the interferon lambda variant for the prevention and treatment of viral infections.

In another aspect, the present invention is directed to a pharmaceutical composition for preventing and treating viral infections comprising the interferon lambda variant.

In another aspect, the present invention is directed to a method for preventing and treating immune diseases comprising administering the interferon lambda variant or a composition comprising the same to a subject.

As used herein, the term “viral infection” means a condition that causes various clinical symptoms such as inflammation, fever, fatigue, chills, vomiting, dizziness, coma or death due to infection with a virus. In the present invention, the virus may be, for example, HCV, HDV, a SARS virus, a MERS virus, an influenza virus, a bird flu virus, or RSV virus, and comprises the latest pandemic SARS-CoV-2 infection (COVID-19), but the invention is not limited thereto.

As herein used, the term “prevention” means any action that inhibits a target disease or delays the progress thereof by administration of the pharmaceutical composition according to the present invention.

As herein used, the term “treatment” refers to any action causing amelioration in symptoms of a target disease or beneficial alteration of the symptoms by administration of the pharmaceutical composition according to the present invention.

The pharmaceutical composition of the present invention exhibits a preventive or therapeutic effect on various viral infections and immune-related diseases based on the antiviral effect and immune function enhancement effect of the interferon lambda variant, which is an active ingredient. In particular, the present compound and composition are used to treat, prevent or slow various viral infections such as mammalian viral infections, particularly, infections with human virus such as HCV, HDV, SARS and MERS.

In addition to the interferon lambda variant, the pharmaceutical composition may further comprise a suitable carrier, vehicle and diluent that are commonly used in pharmaceutical compositions.

Examples of the carrier, vehicle and diluent that may be comprised in the composition may comprise lactose, dextrose, sucrose, sorbitol, mannitol, xylitol, erythritol, maltitol, starch, acacia rubber, alginate, gelatin, calcium phosphate, calcium silicate, cellulose, methylcellulose, microcrystalline cellulose, polyvinylpyrrolidone, water, methylhydroxybenzoate, propylhydroxybenzoate, talc, magnesium stearate, mineral oil and the like. With regard to formulation of the composition, a typically used diluent or vehicle, such as a filler, an extender, a binder, a wetting agent, a disintegrant or a surfactant, is used.

The pharmaceutical composition according to the present invention can be formulated and used in various forms according to a conventional method. Suitable formulations comprise oral formulations such as tablets, pills, powders, granules, dragées, hard or soft capsules, solutions, suspensions, emulsions, and aerosols, injections external preparations, suppositories, sterile injectable solutions, and the like, but are not limited thereto.

The pharmaceutical composition according to the present invention can be prepared into a suitable formulation using a pharmaceutically inactive organic or inorganic carrier. That is, when the formulation is a tablet, a coated tablet, a dragée or a hard capsule, it may comprise lactose, sucrose, starch or a derivative thereof, talc, calcium carbonate, gelatin, stearic acid, or a salt thereof. In addition, when the formulation is a soft capsule, it may comprise a vegetable oil, wax, fat, or semi-solid or liquid polyol. In addition, when the formulation is in the form of a solution or syrup, it may comprise water, polyol, glycerol, vegetable oil or the like.

The pharmaceutical composition according to the present invention may further comprise a preservative, a stabilizer, a wetting agent, an emulsifier, a solubilizing agent, a flavoring agent, a colorant, an osmotic pressure regulator, an antioxidant, or the like, in addition to the above carrier.

The pharmaceutical composition according to the present invention may be administered in a pharmaceutically effective amount, and the term “pharmaceutically effective amount” refers to an amount sufficient for treating a disease at a reasonable benefit/risk ratio applicable to all medical treatments, and the effective dosage level may be determined depending on a variety of factors comprising the type of the disease of the patient, the severity of the disease, the activity of the drug, the sensitivity of the patient to the drug, the administration time, the administration route, the excretion rate, the treatment period, drugs used concurrently therewith, and other factors well-known in the pharmaceutical field. The pharmaceutical composition of the present invention may be administered as a single therapeutic agent or in combination with other therapeutic agents, may be administered sequentially or simultaneously with a conventional therapeutic agent, and may be administered in one or multiple doses. Taking into consideration these factors, it is important to administer the minimum amount sufficient to achieve maximum efficacy without side effects and the amount can be easily determined by those skilled in the art.

The variant or composition according to the present invention may be administered together with other conventional compounds or compositions known to have antiviral and immune-enhancing effects, and may be administered together with other compounds or compositions having an immunosuppressive effect to prevent side effects caused by the composition of the present invention.

The variant or composition according to the present invention may be co-administered with various antiviral agents or adjuvants when used for antiviral application, and may be co-administered with other vaccines and therapeutic agents (e.g., remdesivir or nafamostat) and neutralizing antibodies, for example, when used or administered for the prevention and treatment of COVID-19.

The variant or pharmaceutical composition according to the present invention may be administered to a subject by various routes. The mode of administration may be, for example, subcutaneous, intravenous, intramuscular or intrauterine dural or cerebrovascular injection. The pharmaceutical composition of the present invention is determined according to the type of drug as the active ingredient, as well as various related factors such as the type of the disease to be treated, the route of administration, the age, gender and weight of the patient, and the severity of the disease.

The method of administering the pharmaceutical composition according to the present invention may be easily selected depending on the formulation, and may be administered orally or parenterally. The dosage may vary depending on the age, gender and weight of the patient, severity of the disease, and route of administration.

Since the interferon lambda variant, immunomodulatory composition or pharmaceutical composition according to the present invention exhibits a remarkable immunity-enhancing effect, it can be used for the prevention and treatment of various diseases caused by deteriorated or abnormal immunity or having the same as a symptom, as well as viral infections.

Accordingly, in another aspect, the present invention is directed to the use of the interferon lambda variant of the present invention for the prevention and treatment of various diseases caused by deteriorated or abnormal immunity or having the same as a symptom, in addition to viral infections.

In another aspect, the present invention is directed to a pharmaceutical composition for preventing and treating of various diseases caused by deteriorated or abnormal immunity or having the same as a symptom, in addition to viral infections.

In another aspect, the present invention is directed to a method for preventing and treating various diseases caused by deteriorated or abnormal immunity or having the same as a symptom, in addition to viral infections, the method comprising administering to a subject the interferon lambda variant or the composition comprising the same according to the present invention.

As herein used, the term “various diseases caused by deteriorated or abnormal immunity or having the same as a symptom, in addition to viral infections” comprise, for example, cancer, tumors, organ transplant rejection, chronic kidney failure, cirrhosis, diabetes and hyperglycemia, but are not limited thereto.

Since the interferon lambda 4 variant according to the embodiment of the present invention was produced through a structure-based design so as to maintain the conserved sequence of the conventional interferon lambda 1 to 4, the method of producing the interferon lambda variant according to the present invention is not limited to interferon lambda 4, and can be used to produce interferon lambda that exhibits expression ability, therapeutic properties and biological activity superior to those of a type III interferon, and in particular, can also be used for interferon lambda 3 based on the very similar binding model structure.

Advantageously, the method of producing the interferon lambda variant according to the present invention is capable of producing interferon lambda variants having superior expression ability, therapeutic properties and biological activity despite inducing small mutations through structure-based screening of mutation sites, among a number of interferon lambda mutation sites, and is useful for deriving the screening of potential mutation sites.

Accordingly, in another aspect, the present invention is directed to a method of producing an interferon lambda variant comprising culturing the recombinant microorganism or the recombinant cell to express an interferon lambda variant, and collecting the expressed interferon lambda variant.

In the present invention, the interferon lambda variant produced by the production method described above may share the same characteristics and embodiments as described above.

In the present invention, the method of inducing mutation for the interferon lambda, the method for producing a recombinant cell, the expression and purification methods according to the present invention described in Examples, and the like are provided only as exemplary embodiments, and can be easily implemented without limitation through the conventionally known invention that can be selected by those skilled in the art.

### EXAMPLE

Hereinafter, the present invention will be described in more detail with reference to examples. However, it will be obvious to those skilled in the art that these examples are provided only for illustration of the present invention and should not be construed as limiting the scope of the present invention.

### Example 1: Materials and Methods

### Example 1-1: IL10Rβ-IFNλ4-IFNλR1 Modeling

The human IFNλ4 amino acid sequence (22˜179, NCBI Accession Number: AFQ38559.1) was used in SWISS-MODEL homology modeling with three templates (PDB code: 5T5W.1.C, 30G6.1.A, 30G4.1.A). The model with the highest QMEAN-Z (Qualitative Model Energy ANalysis-Z) score (−2.56) was aligned to the IL10Rβ-IFNλ3-IFNλR1 structure (PDB code: 5T5W) to create the IL10Rβ-IFNλ4-IFNλR1 model.

### Example 1-2: Cell Line, Cell Culture and Reagent

Expi293F (#A14527, Gibco®) cells were cultured according to ATCC guidelines and used within 6 months of receipt. They were maintained in suspension in Expi293F expression medium (#14351, Gibco®) at 37° C. and 8% CO₂ with 125 rpm agitation. Huh-7.5 cells (Apath) were maintained at 37° C. with 5% CO₂ in Dulbecco's modified Eagle medium (DMEM) containing 10% fetal bovine serum (WelGENE), 4.5g/l glucose, L-glutamine, and 1% penicillin/streptomycin (WelGENE). Small-interfering RNAs (siRNAs) against IFNλR1 and scrambled sequences were obtained from Santa Cruz Biotechnology. Transfection of IFNλR1 siRNA was performed using lipofectamine RNAi MAX (Invitrogen). Recombinant IFN-α-2a was obtained from PBL Assay Science, recombinant IFN-β was obtained from PeproTech, and recombinant human IFNλ1 (1598-IL), λ2 (8417-IL), λ3 (5259-IL), and eIFNλ4 (9165-IL) were obtained from R&D Systems. The eIFNλ4 produced in E. coli was used as control wild-type IFNλ4.

### Example 1-3: Expression and Purification of Recombinant Protein

Gene encoding human IFNλ4 (1˜179) was cloned into a modified pcDNA3.1 (#V79020, Invitrogen™) containing a C-terminal 6×-His tag. IFNλ4 variants were generated by site-directed mutagenesis (QuikChange site-Directed Mutagenesis Kit, #200519, Agilent) using the IFNλ4 wild-type construct as the PCR template. The primers for site-directed mutagenesis are listed in Table 1.

TABLE 1
Primers for site-directed mutagenesis

| Mutation | | | SEQ ID |
|---|---|---|---|
| site | | Sequence 5′→3′ | NO |
| L28N | Forward | CAGCCCCTAGAAGATGCAATCTCTCCCACTACCGCA | 1 |
| (m1) | primer | G | |
| | Reverse | CTGCGGTAGTGGGAGAGATTGCATCTTCTAGGGGCT | 2 |
| | primer | G | |
| A54N | Forward | GGACAGATATGAGGAAGAAAACCTGAGCTGGGGGC | 3 |
| (m2) | primer | A | |
| | Reverse | TGCCCCCAGCTCAGGTTTTCTTCCTCATATCTGTCC | 4 |
| | primer | | |
| P73N | Forward | GAGGGACCCTCCAAGGAATAGCTCCTGCGCTAGGC | 5 |
| (m3) | primer | | |
| | Reverse | GCCTAGCGCAGGAGCTATTCCTTGGAGGGTCCCTC | 6 |
| | primer | | |
| H97N | Forward | GTGCTTAGCGGCCTTAACAGGTCCGAGCT | 7 |
| (m4) | primer | | |
| | Reverse | AGCTCGGACCTGTTAAGGCCGCTAAGCAC | 8 |
| | primer | | |
| K154N | Forward | CCCCGGTGTCGGAATGCCTCTGTGGT | 9 |
| (m5) | primer | | |
| | Reverse | ACCACAGAGGCATTCCGACACCGGGG | 10 |
| | primer | | |
| A173N | Forward | GTTGCGCTTGGCCAACCACAGCGGGCCC | 11 |
| (m6) | primer | | |
| | Reverse | GGGCCCGCTGTGGTTGGCCAAGCGCAAC | 12 |
| | primer | | |

*Lines mean mutation sites

A double mutation (L28N+P73N) was induced in M7 by simultaneously using the primers for M1 and M3 mutagenesis in the above table.

For IFNλ4-Protein A expression, the C-terminal 6×-His in the IFNλ4 constructs were replaced with a Protein A gene derived from PEZZ18 (#VPT4033, GE Healthcare life Sciences). For protein A removal, a thrombin cleavage sequence (LVPRGS) was introduced between the IFNλ4 gene and the Protein A gene using PCR primers. IFNλ4 wild-type and variants containing 6×-His or Protein A were transfected into Expi293F cells using ExpiFectamine 293 Transfection Kits (#A14524, Invitrogen™) according to the manufacturer's protocol. For the purification of IFNλ4 variants, the supernatant containing secreted IFNλ4-Protein A was loaded onto IgG Sepharose resin (#17096902, GE Healthcare Life Sciences). After three washes with 1× PBS, the protein-bound resins were incubated overnight with thrombin (1% (v/v) in 1× PBS) at 4° C. to remove the C-terminal Protein A tag. Eluted IFNλ4 variants were subsequently purified by gel-filtration chromatography in a Superdex 200 Increase 10/300 GL column (#28990944, GE Healthcare Life Sciences) equilibrated with 1× PBS.

### Example 1-4: Immunoblotting

The cells were lysed with RIPA buffer (Thermo Fisher Scientific) to prepare a total cell lysate. 10 μg of each cell lysate was loaded onto the SDS-PAGE gel before immunoblotting. The antibodies used for the immunoblotting were as follows: IFNλ4 (1:200, mouse, Millipore MABF227), IFNλ4 (1:200, rabbit, Abcam ab196984), STAT1 (1:1000, rabbit, BD Biosciences 610120), PY-STAT1 (1:1000, mouse, BD Biosciences 612233), STAT2 (1:1000, rabbit, Santa Cruz Biotechnology sc-476), IRF9 (1:1000, rabbit, Santa Cruz sc-496), SOCS1 (Abcam #62584), USP18 (Cell Signaling Technology #4813), HRP (horseradish peroxidase)-conjugated rabbit IgG (1:5000, Abcam ab97051), and HRP-conjugated mouse IgG (1:5000, Abcam ab97023).

### Example 1-5: PNGase F Treatment

N-glycans of IFNλ4 were removed using a PNGase F kit (#P0704S, New England Biolabs) according to the manufacturer's instructions. Specifically, the IFNλ4 variant was boiled in a glycoprotein denaturing buffer (10×) and cooled on ice. GlycoBuffer (10×), NP-40 (10×) and 1 μl of PNGase F were added to the denatured protein and the mixture was incubated at 37° C. for 1 hour before Western blot analysis.

### Example 1-6: Analysis of Glycosylation Site

Glycopeptides produced by non-specific digestion were prepared by a known method (Journal of proteome research 2013;12:4414-23). Specifically, 50 μg/pl of the IFNλ4 variant was incubated with 50 μg/pl of Pronase E for 1 hour at 37° C. The digested glycopeptides were enriched by graphitized carbon solid-phase extraction (PGC-SPE) and analyzed by nanoLC-Chip Q-TOF MS (Agilent Technologies). LC-MS and MS/MS data were processed and interpreted with MAssHunter Qualitative Analysis software (version B.07.00, Agilent Technologies) and GP Finder software (Journal of Proteome Research 2006;5:2800-8).

### Example 1-7: Confirmation of Binding Kinetics

The binding kinetics of the IFNλ4 variant to IFNλR1 and IL10Rβ were measured using biolayer light interferometry (BLI) in a BLItz system (ForteBio, Pall Life Sciences). The mixture was stirred in a washing buffer (200 mM NaCl, 20 mM Tris-HCl pH 8, 5% glycerol, 0.01% Tween-20) at 2,200 rpm. Analysis was performed at room temperature. 0.25 mg/ml of biotinylated IFNλ4 was loaded on the surface of the streptavidin biosensor (ForteBio) for 1 minute, and then the loaded biosensor was washed with a washing buffer (200 mM NaCl, 20 mM Tris-HCl pH 8, 5% glycerol, 0.01% Tween-20) for 2 minutes to remove unbound protein. The biosensor tip was immersed in drops containing the indicated concentrations of IFNλR1 and IL10Rβ (500, 1000 and 2000 nM). Associations (on rate, kon) were measured at intervals of 2 minutes. Subsequently, the sensor was immersed in the washing buffer for 2 minutes to measure dissociations (off-rate koff). K_{D} measured in nanomoles was calculated as a ratio of on-rate to off-rate. The resulting data was analyzed by fitting to a 1:1 ligand model using the entire global fitting function.

### Example 1-8: Production and Infection of HCV-Derived Cell Culture (HCVcc)

The Japanese fulminant hepatits-1 (JFH-1) strain (genotype 2a) of HCVcc was produced by the method described above (Proc. Natl. Acad. Sci. USA 2015;112:10443-8). DMEM containing 5% human serum was used to culture Huh-7.5 cells for production of infectious JFH1 HCVcc. HCVcc infectivity was quantified by a colorimetric focus-forming assay (PLoS One 2012;7:e43960), which is a previously published method. The Huh-7.5 cells were infected with JFH-1 HCVcc at 0.5 MOI (multiplicity of infection).

### Example 1-9: RNA Extraction and Real-Time Quantitative PCR

Total RNA isolation and TaqMan real-time quantitative PCR were performed by a conventionally known method. In brief, total RNA was isolated with GeneAll Ribospin™ (GeneAll), after which TaqMan Gene Expression Assays (Applied Biosystems) were used to determine the mRNA levels of the target genes. Quantification of intracellular HCV RNA copies was performed as described previously (Journal of virology 2014;88:9233-44). The results were standardized to the mRNA levels of GAPDH and the data are presented as means±standard error of the mean. TaqMan Assay (Applied Biosystems) used in this study are: IFNLR1 (Hs00417120_m1), ISG15 (Hs01921425 _s1), MX1 (Hs00895608 _m1), SOCS1 (Hs00705164 _s1), USP18 (Hs00276441 _m1), GAPDH (Hs02758991 _g1). IFNL proteins (R&D Systems) used in this study are: IFNL1 (1598-IL), IFNL2 (8417-IL), IFNL3 (5259-IL), eIFNL4 (9165-IL).

### Example 1-10: Statistical Analysis

Data from experiments using cell lines are represented as mean±standard error (SE). For statistical analysis, an unpaired t-test or a two-tailed Mann-Whitney U-test was performed. All real-time quantitative PCR analysis was performed with GraphPad Prism version 7.01, and a P value less than 0.05 was determined to be a statistically significant.

### Example 2: Design and Expression of IFNλ4 Variants

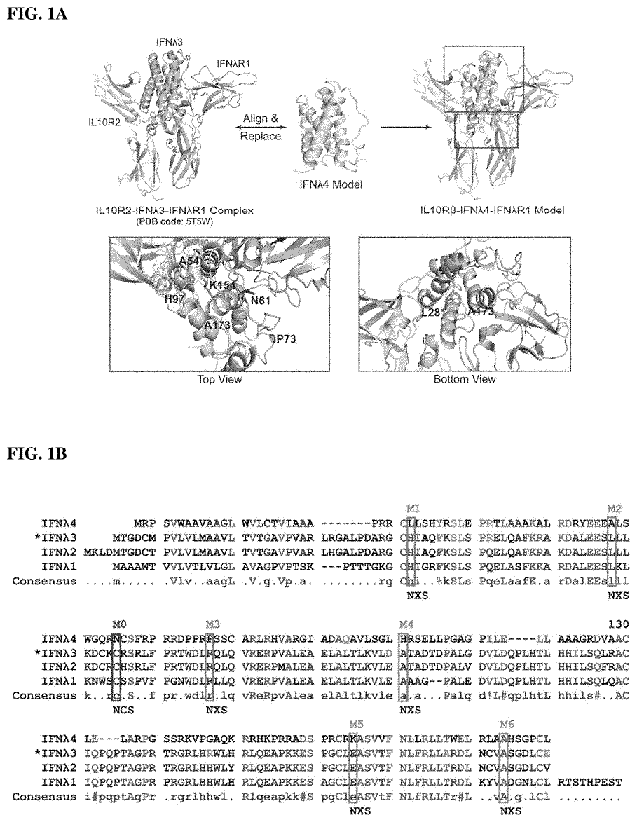

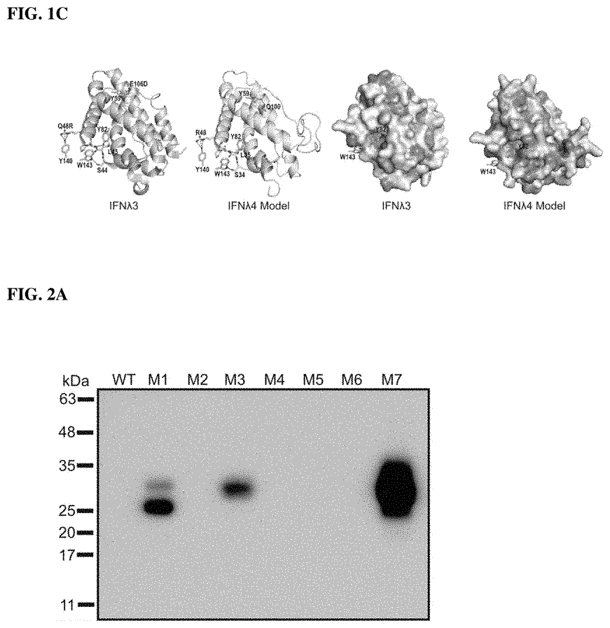

The low affinity for the receptor of wild-type IFNλ inhibits the production of stable 3-complex-IL10Rβ-IFNλ-IFNλR1. Therefore, only the structure of IFNλ3 (The Journal of Biological Chemistry 2009;284:20869-75) or IFNλ1 (J. Mol. Biol. 2010;404:650-64) was detected in the complex with IFNλR1. Recently, Mendoza, et al. have introduced an affinity-enhanced mutation into IFNλ3 to stabilize the interaction with IL10Rβ, and described the crystal structure of the type III interferon signaling complex IL10Rβ-IFNλ3-IFNλR1 (PDB code: 5T5W) (FIG. 1A). IFNλ4 shares about 30% sequence identity with IFNλ1 to IFNλ3, but the results of the sequence alignment of IFNλ1 to IFNλ4 suggest that IFNλ4 interacts with IFNλR1 and IL10Rβ in a manner similar to the IL10Rβ-IFNλ3-IFNλR1 ternary complex (Immunity 2017;46:379-92) for the following two reasons. First, amino acids of the IFNλ family, which are important for IFNλR1 binding, are well conserved in IFNλ4 (P37, L40, K44, R47, D48, I108, F159 and R163) (FIG. 1B). Second, the hydroxyl groups of several aromatic moieties (Y59, Y82, Y140 and W143) of IL10Rβ form a hydrogen-bonding network with IFNλ3 (S44, L45, Q48R and E106D), and they are also well conserved in IFNλ4 (S34, L35, R48 and Q100). Thus, the structure of IFNλ4 was modeled using the crystal structures of IFNλ3 and IFNλ1 (FIG. 1A), and an IL10Rβ-IFNλ4-IFNλR1 model was constructed by aligning the IL10Rβ-IFNλ3-IFNλR1 structure (FIG. 1B). Interestingly, the model structure of IL10Rβ-IFNλ4-IFNλR1 shows that major hydrophobic pockets harboring hydrophobic residues (Y82 and W143) of IL10Rβ are well maintained on the surface of IFNλ4 (FIG. 1C).

Novel N-glycosylation candidate sites of IFNλ4 were screened based on the following three criteria using the IL10Rβ-IFNλ4-IFNλR1 model structure.

First, the sites had to be outside the receptor binding region to minimize the change in the receptor-ligand binding and signal activation.

Second, they had to be exposed to the solvent to allow access to oligosaccharyltransferase (OST), which catalyzes the initial transfer of glycan from the lipid-linked oligosaccharide onto the substrate asparagine;

Third, the consensus sequence (NXS/T, X=any amino acid except proline) had to be achieved by single point mutation to minimize the structural distortion caused by the mutation.

Only six sites satisfying all the three criteria, namely L28, A54, P73, H97, K154 and A173, were identified (FIGS. 1A and 1B) and were named as m1 to m6, respectively.

Variants (M1 to M6) in which the amino acids at positions m1 to m6 were substituted with asparagine (N) were produced using the primers shown in Table 1 of Example 1-3, and a double variant (M7) in which both L28 (m1) and P73 (m3) are substituted with asparagine (N) was produced. The gene sequence and amino acid sequence of each of the produced interferon lambda variants (M1 to M7) are shown in Tables 2 and 3, respectively.

TABLE 2
Gene sequences encoding interferon lambda variants

| | | SEQ |
|---|---|---|
| Mutation | | ID |
| site | Gene sequence 5′→3′ | NO |
| eIFNλ4 | Purchased in protein form | — |
| L28N | ATGAGACCTTCCGTCTGGGCCGCCGTGGCCGCAGGACT | 13 |
| (M1) | GTGGGTCCTGTGCACCGTGATCGCCGCAGCCCCTAGAA | |
| | GATGCAATCTCTCCCACTACCGCAGCCTGGAGCCCAGA | |
| | ACACTGGCCGCTGCCAAGGCCCTGAGGGACAGATATGA | |
| | GGAAGAAGCCCTGAGCTGGGGGCAGCGGAACTGCTCCT | |
| | TCCGGCCCAGGAGGGACCCTCCAAGGCCAAGCTCCTGC | |
| | GCTAGGCTCAGGCACGTGGCTAGGGGAATCGCCGACGC | |
| | TCAGGCTGTGCTTAGCGGCCTTCACAGGTCCGAGCTGCT | |
| | CCCTGGCGCTGGCCCAATTCTGGAGCTGCTGGCCGCAGC | |
| | AGGGAGGGATGTGGCCGCCTGTCTTGAGTTGGCCAGGC | |
| | CAGGCTCTAGTCGGAAGGTCCCCGGGGCCCAAAAGCGG | |
| | CGCCATAAACCCCGGCGGGCCGATTCACCCCGGTGTCG | |
| | GAAGGCCTCTGTGGTCTTTAATCTCCTCCGGCTCCTGAC | |
| | TTGGGAGTTGCGCTTGGCCGCCCACAGCGGGCCCTGCTT | |
| | G | |
| A54N | ATGAGACCTTCCGTCTGGGCCGCCGTGGCCGCAGGACT | 14 |
| (M2) | GTGGGTCCTGTGCACCGTGATCGCCGCAGCCCCTAGAA | |
| | GATGCCTGCTCTCCCACTACCGCAGCCTGGAGCCCAGAA | |
| | CACTGGCCGCTGCCAAGGCCCTGAGGGACAGATATGAG | |
| | GAAGAAAACCTGAGCTGGGGGCAGCGGAACTGCTCCTT | |
| | CCGGCCCAGGAGGGACCCTCCAAGGAATAGCTCCTGCG | |
| | CTAGGCTCAGGCACGTGGCTAGGGGAATCGCCGACGCT | |
| | CAGGCTGTGCTTAGCGGCCTTCACAGGTCCGAGCTGCTC | |
| | CCTGGCGCTGGCCCAATTCTGGAGCTGCTGGCCGCAGCA | |
| | GGGAGGGATGTGGCCGCCTGTCTTGAGTTGGCCAGGCC | |
| | AGGCTCTAGTCGGAAGGTCCCCGGGGCCCAAAAGCGGC | |
| | GCCATAAACCCCGGCGGGCCGATTCACCCCGGTGTCGG | |
| | AAGGCCTCTGTGGTCTTTAATCTCCTCCGGCTCCTGACTT | |
| | GGGAGTTGCGCTTGGCCGCCCACAGCGGGCCCTGCTTG | |
| P73N | ATGAGACCTTCCGTCTGGGCCGCCGTGGCCGCAGGACT | 15 |
| (M3) | GTGGGTCCTGTGCACCGTGATCGCCGCAGCCCCTAGAA | |
| | GATGCCTGCTCTCCCACTACCGCAGCCTGGAGCCCAGAA | |
| | CACTGGCCGCTGCCAAGGCCCTGAGGGACAGATATGAG | |
| | GAAGAAGCCCTGAGCTGGGGGCAGCGGAACTGCTCCTT | |
| | CCGGCCCAGGAGGGACCCTCCAAGGAATAGCTCCTGCG | |
| | CTAGGCTCAGGCACGTGGCTAGGGGAATCGCCGACGCT | |
| | CAGGCTGTGCTTAGCGGCCTTCACAGGTCCGAGCTGCTC | |
| | CCTGGCGCTGGCCCAATTCTGGAGCTGCTGGCCGCAGCA | |
| | GGGAGGGATGTGGCCGCCTGTCTTGAGTTGGCCAGGCC | |
| | AGGCTCTAGTCGGAAGGTCCCCGGGGCCCAAAAGCGGC | |
| | GCCATAAACCCCGGCGGGCCGATTCACCCCGGTGTCGG | |
| | AAGGCCTCTGTGGTCTTTAATCTCCTCCGGCTCCTGACTT | |
| | GGGAGTTGCGCTTGGCCGCCCACAGCGGGCCCTGCTTG | |
| H97N | ATGAGACCTTCCGTCTGGGCCGCCGTGGCCGCAGGACT | 16 |
| (M4) | GTGGGTCCTGTGCACCGTGATCGCCGCAGCCCCTAGAA | |
| | GATGCCTGCTCTCCCACTACCGCAGCCTGGAGCCCAGAA | |
| | CACTGGCCGCTGCCAAGGCCCTGAGGGACAGATATGAG | |
| | GAAGAAGCCCTGAGCTGGGGGCAGCGGAACTGCTCCTT | |
| | CCGGCCCAGGAGGGACCCTCCAAGGCCAAGCTCCTGCG | |
| | CTAGGCTCAGGCACGTGGCTAGGGGAATCGCCGACGCT | |
| | CAGGCTGTGCTTAGCGGCCTTAACAGGTCCGAGCTGCTC | |
| | CCTGGCGCTGGCCCAATTCTGGAGCTGCTGGCCGCAGCA | |
| | GGGAGGGATGTGGCCGCCTGTCTTGAGTTGGCCAGGCC | |
| | AGGCTCTAGTCGGAAGGTCCCCGGGGCCCAAAAGCGGC | |
| | GCCATAAACCCCGGCGGGCCGATTCACCCCGGTGTCGG | |
| | AAGGCCTCTGTGGTCTTTAATCTCCTCCGGCTCCTGACTT | |
| | GGGAGTTGCGCTTGGCCGCCCACAGCGGGCCCTGCTTG | |
| K154N | ATGAGACCTTCCGTCTGGGCCGCCGTGGCCGCAGGACT | 17 |
| (M5) | GTGGGTCCTGTGCACCGTGATCGCCGCAGCCCCTAGAA | |
| | GATGCCTGCTCTCCCACTACCGCAGCCTGGAGCCCAGAA | |
| | CACTGGCCGCTGCCAAGGCCCTGAGGGACAGATATGAG | |
| | GAAGAAGCCCTGAGCTGGGGGCAGCGGAACTGCTCCTT | |
| | CCGGCCCAGGAGGGACCCTCCAAGGCCAAGCTCCTGCG | |
| | CTAGGCTCAGGCACGTGGCTAGGGGAATCGCCGACGCT | |
| | CAGGCTGTGCTTAGCGGCCTTCACAGGTCCGAGCTGCTC | |
| | CCTGGCGCTGGCCCAATTCTGGAGCTGCTGGCCGCAGCA | |
| | GGGAGGGATGTGGCCGCCTGTCTTGAGTTGGCCAGGCC | |
| | AGGCTCTAGTCGGAAGGTCCCCGGGGCCCAAAAGCGGC | |
| | GCCATAAACCCCGGCGGGCCGATTCACCCCGGTGTCGG | |
| | AATGCCTCTGTGGTCTTTAATCTCCTCCGGCTCCTGACTT | |
| | GGGAGTTGCGCTTGGCCGCCCACAGCGGGCCCTGCTTG | |
| A173N | ATGAGACCTTCCGTCTGGGCCGCCGTGGCCGCAGGACT | 18 |
| (M6) | GTGGGTCCTGTGCACCGTGATCGCCGCAGCCCCTAGAA | |
| | GATGCCTGCTCTCCCACTACCGCAGCCTGGAGCCCAGAA | |
| | CACTGGCCGCTGCCAAGGCCCTGAGGGACAGATATGAG | |
| | GAAGAAGCCCTGAGCTGGGGGCAGCGGAACTGCTCCTT | |
| | CCGGCCCAGGAGGGACCCTCCAAGGCCAAGCTCCTGCG | |
| | CTAGGCTCAGGCACGTGGCTAGGGGAATCGCCGACGCT | |
| | CAGGCTGTGCTTAGCGGCCTTCACAGGTCCGAGCTGCTC | |
| | CCTGGCGCTGGCCCAATTCTGGAGCTGCTGGCCGCAGCA | |
| | GGGAGGGATGTGGCCGCCTGTCTTGAGTTGGCCAGGCC | |
| | AGGCTCTAGTCGGAAGGTCCCCGGGGCCCAAAAGCGGC | |
| | GCCATAAACCCCGGCGGGCCGATTCACCCCGGTGTCGG | |
| | AAGGCCTCTGTGGTCTTTAATCTCCTCCGGCTCCTGACTT | |
| | GGGAGTTGCGCTTGGCCAACCACAGCGGGCCCTGCTTG | |
| L28N + P73N | ATGAGACCTTCCGTCTGGGCCGCCGTGGCCGCAGGACT | 19 |
| (M7) | GTGGGTCCTGTGCACCGTGATCGCCGCAGCCCCTAGAA | |
| | GATGCAATCTCTCCCACTACCGCAGCCTGGAGCCCAGA | |
| | ACACTGGCCGCTGCCAAGGCCCTGAGGGACAGATATGA | |
| | GGAAGAAGCCCTGAGCTGGGGGCAGCGGAACTGCTCCT | |
| | TCCGGCCCAGGAGGGACCCTCCAAGGAATAGCTCCTGC | |
| | GCTAGGCTCAGGCACGTGGCTAGGGGAATCGCCGACGC | |
| | TCAGGCTGTGCTTAGCGGCCTTCACAGGTCCGAGCTGCT | |
| | CCCTGGCGCTGGCCCAATTCTGGAGCTGCTGGCCGCAGC | |
| | AGGGAGGGATGTGGCCGCCTGTCTTGAGTTGGCCAGGC | |
| | CAGGCTCTAGTCGGAAGGTCCCCGGGGCCCAAAAGCGG | |
| | CGCCATAAACCCCGGCGGGCCGATTCACCCCGGTGTCG | |
| | GAAGGCCTCTGTGGTCTTTAATCTCCTCCGGCTCCTGAC | |
| | TTGGGAGTTGCGCTTGGCCGCCCACAGCGGGCCCTGCTT | |
| | G | |

*Bold lines mean mutation sites

TABLE 3
Amino acid sequences of wild-type interferon lambda 4
and variants thereof

| | | SEQ |
|---|---|---|
| Mutation | | ID |
| sites | Amino acid sequence N′→C′ | NO |
| IFNλ4 | MRPSVWAAVAAGLWVLCTVIAAAPRRCLLSHYRSLEPRT | 20 |
| (wild-type) | LAAAKALRDRYEEEALSWGQRNCSFRPRRDPPRPSSCARL | |
| | RHVARGIADAQAVLSGLHRSELLPGAGPILELLAAAGRDV | |
| | AACLELARPGSSRKVPGAQKRRHKPRRADSPRCRKASVVF | |
| | NLLRLLTWELRLAAHSGPCL | |
| eIFNλ4 | MAPRRCLLSHYRSLEPRTLAAAKALRDRYEEEALSWGQR | 21 |
| | NCSFRPRRDPPRPSSCARLRHVARGIADAQAVLSGLHRSEL | |
| | LPGAGPILELLAAAGRDVAACLELARPGSSRKVPGAQKRR | |
| | HKPRRADSPRCRKASVVFNLLRLLTWELRLAAHSGPCL | |
| L28N | MRPSVWAAVAAGLWVLCTVIAAAPRRCNLSHYRSLEPRT | 22 |
| (M1) | LAAAKALRDRYEEEALSWGQRNCSFRPRRDPPRPSSCARL | |
| | RHVARGIADAQAVLSGLHRSELLPGAGPILELLAAAGRDV | |
| | AACLELARPGSSRKVPGAQKRRHKPRRADSPRCRKASVVF | |
| | NLLRLLTWELRLAAHSGPCL | |
| A54N | MRPSVWAAVAAGLWVLCTVIAAAPRRCLLSHYRSLEPRT | 23 |
| (M2) | LAAAKALRDRYEEENLSWGQRNCSFRPRRDPPRNSSCARL | |
| | RHVARGIADAQAVLSGLHRSELLPGAGPILELLAAAGRDV | |
| | AACLELARPGSSRKVPGAQKRRHKPRRADSPRCRKASVVF | |
| | NLLRLLTWELRLAAHSGPCL | |
| P73N | MRPSVWAAVAAGLWVLCTVIAAAPRRCLLSHYRSLEPRT | 24 |
| (M3) | LAAAKALRDRYEEEALSWGQRNCSFRPRRDPPRNSSCARL | |
| | RHVARGIADAQAVLSGLHRSELLPGAGPILELLAAAGRDV | |
| | AACLELARPGSSRKVPGAQKRRHKPRRADSPRCRKASVVF | |
| | NLLRLLTWELRLAAHSGPCL | |
| H97N | MRPSVWAAVAAGLWVLCTVIAAAPRRCLLSHYRSLEPRT | 25 |
| (M4) | LAAAKALRDRYEEEALSWGQRNCSFRPRRDPPRPSSCARL | |
| | RHVARGIADAQAVLSGLNRSELLPGAGPILELLAAAGRDV | |
| | AACLELARPGSSRKVPGAQKRRHKPRRADSPRCRKASVVF | |
| | NLLRLLTWELRLAAHSGPCL | |
| K154N | MRPSVWAAVAAGLWVLCTVIAAAPRRCLLSHYRSLEPRT | 26 |
| (M5) | LAAAKALRDRYEEEALSWGQRNCSFRPRRDPPRPSSCARL | |
| | RHVARGIADAQAVLSGLHRSELLPGAGPILELLAAAGRDV | |
| | AACLELARPGSSRKVPGAQKRRHKPRRADSPRCRNASVVF | |
| | NLLRLLTWELRLAAHSGPCL | |
| A173N | MRPSVWAAVAAGLWVLCTVIAAAPRRCLLSHYRSLEPRT | 27 |
| (M6) | LAAAKALRDRYEEEALSWGQRNCSFRPRRDPPRPSSCARL | |
| | RHVARGIADAQAVLSGLHRSELLPGAGPILELLAAAGRDV | |
| | AACLELARPGSSRKVPGAQKRRHKPRRADSPRCRKASVVF | |
| | NLLRLLTWELRLANHSGPCL | |
| L28N + P73N | MRPSVWAAVAAGLWVLCTVIAAAPRRCNLSHYRSLEPRT | 28 |
| (M7) | LAAAKALRDRYEEEALSWGQRNCSFRPRRDPPRNSSCARL | |
| | RHVARGIADAQAVLSGLHRSELLPGAGPILELLAAAGRDV | |
| | AACLELARPGSSRKVPGAQKRRHKPRRADSPRCRKASVVF | |
| | NLLRLLTWELRLAAHSGPCL | |

*Bold lines mean mutation sites

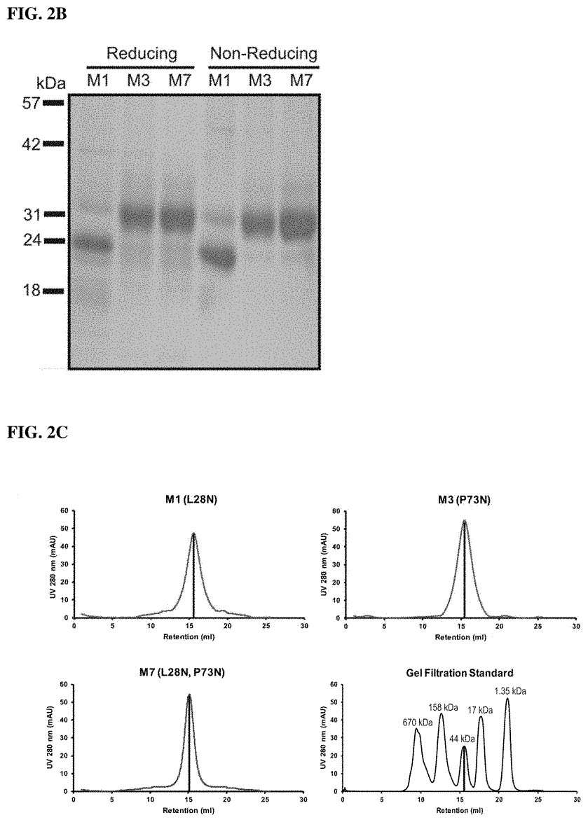

Next, using Western blotting, the expression level of IFNλ4 variants (M1 to M6) was examined, and it was found that protein expression was improved in two IFNλ4 variants, M1 (L28N mutation) and M3 (P73N mutation) (FIG. 2A).

Interestingly, only M3 showed a significant upward shift in SDS-PAGE, which indicates that over-glycosylation proceeded successfully. In addition, the double mutant (L28N and P73N, M7) exhibited improved protein expression compared to the M1 and M3 variants (FIG. 2A). The construct used in Western blotting for hit discovery has a 6× Histidine tag at the C-terminus, which may interfere with proper secretion of the protein when considering a wide range of positively charged amino acids of IFNλ4. Thus, the present inventors replaced the 6× His tag with the Protein A tag for removal of the Protein A tag and subsequent size exclusion chromatography and purified IFNλ4 variants (M1, M3 and M7) using affinity chromatography and thrombin digestion. The final IFNλ4 variants (M1, M3, and M7) were analyzed using SDS-PAGE and Coomassie blue staining under reducing and non-reducing conditions. The resulting bands indicate that the three IFNλ4 variants (M1, M3, and M7) are monomers (FIG. 2B). The elution profile of the standard protein shows that each monodisperse peak corresponds to the IFNλ4 variant (about 44 kDa) (FIG. 2C). In most cases, this oversized elution is due to the presence of N-glycosylation in IFNλ4, which was verified by the results described in Example 3.

### Example 3: N-glycan Identification of IFNλ4 Variants

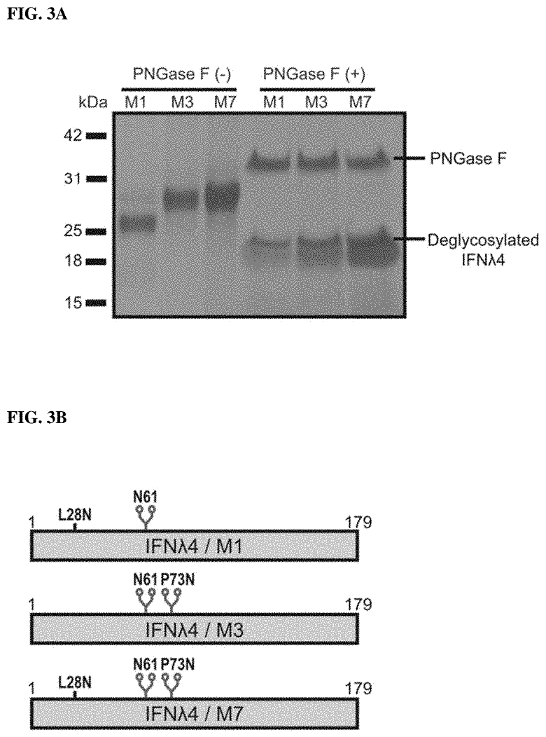

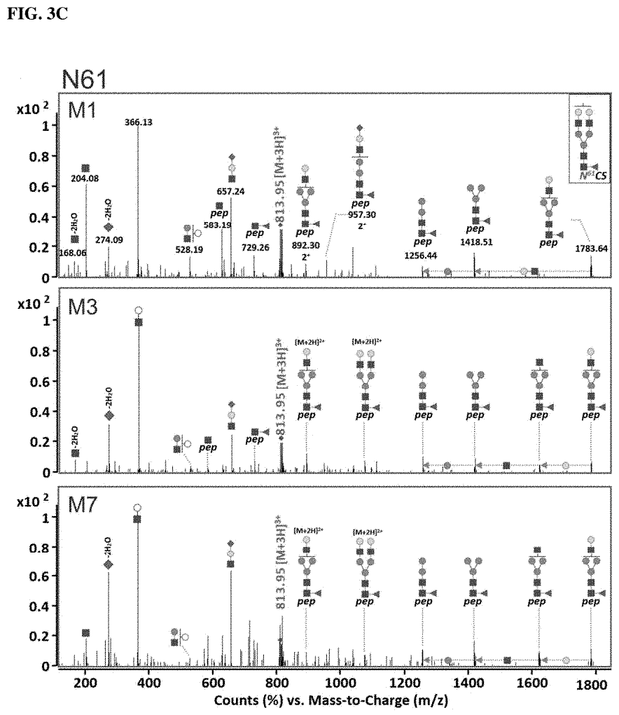

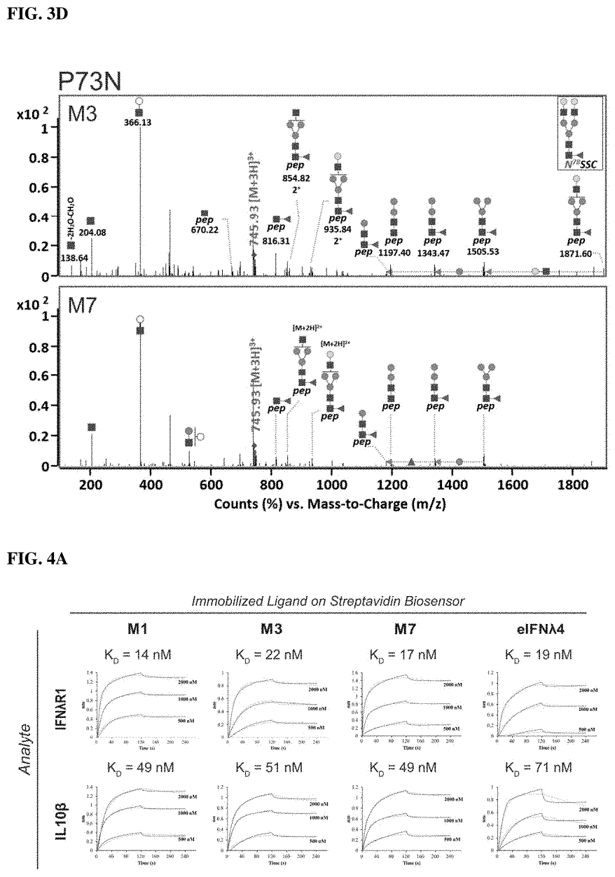

In order to identify the presence of N-glycans in the above three IFNλ4 variants, the N-glycans were treated with PNGase F, and a size comparison was conducted through SDS-PAGE. The M3 (P73N) and M7 (L28N+P73N) IFNλ4 variants exhibited higher molecular weights than the M1 (L28N) IFNλ4 variants. However, after deglycosylation using PNGase F, the molecular weights of the three IFNλ4 variants decreased to the same level, which indicates the presence of N-glycans in all IFNλ4 variants, but the N-glycosylation site of M1 may be slightly different from the N-glycosylation site of M3 and M7 IFNλ4 variants (FIG. 3A).

Mass spectrometry was used to determine the exact location of the N-glycan in the IFNλ4 variant. The purified IFNλ4 variant was treated with Pronase E to produce a glycopeptide, and the glycosylation site was finally determined. Then, the glycopeptide was isolated and analyzed using nanoLC-Chip Q-TOF MS. LC/MS data show that the varied L28N in the M1 and M7 IFNλ4 variants was not glycosylated, whereas the original N-glycosylation site, Asn61, and varied P73N were completely occupied by N-glycans (FIGS. 3B to 3D). This is consistent with the result of PNGase F treatment, which showed that M1 (L28N) shifts faster than M3 (P73N) or M7 (L28N+P73N).

### Example 4: Receptor Binding Affinity and Biological Activity of IFNλ4 Variants

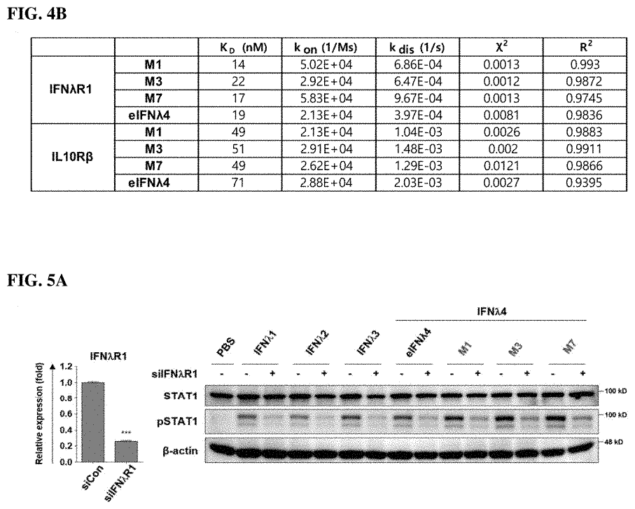

To investigate whether the mutation and the additional glycan on IFNλ4 variants affect their binding to their receptors, IL10Rβ and IFNλR1, in-vitro binding affinity of the IFNλ4 variants to IL10Rβ and IFNλR1 was evaluated using biolayer light interferometry (BLI) and then compared with that of wild-type IFNλ4 (eIFNλ4) purified from E. coli. Similar to eIFNλ4, the three IFNλ4 variants appropriately bound to the receptor, and the binding affinity for IFNλR1 was higher than the binding affinity for IL10Rβ (FIG. 4). In addition, the IFNλ4 variant of the present invention had slightly higher affinity for IL10Rβ than eIFNλ4 (in the case of IL10Rβ, KD M1=49 nM, KD M3=51 nM, KD M7=49 nM, KD eIFNλ4=71 nM), and the binding affinity for IFNλR1 was similar to that of eIFNλ4 (for IFNλR1, KD M1=14 nM, KD M3=22 nM, KD M7=17 nM, KD eIFNλ4=19 nM). Modifications induced by mutations and glycosylation do not inhibit interaction with specific receptors thereof, but further stabilize the interaction between IFNλ4 and IL10Rβ.

In order to determine whether or not mutations and additional glycans in the IFNλ4 variant affect functional activity, the present inventors detected IFNλR1-dependent phospho-STAT1 signaling upon treatment with the IFNλ4 variant. The result showed that, similar to IFNλ1 to IFNλ3, which are other type III interferons, M1, M3 and M7 IFNλ4 variants induced phosphorylation of STAT1 and phosphorylation of STAT1 was blocked, in spite of treatment with the IFNλ4 variants, when inhibiting the expression of IFNλR1 with a small interfering RNA (siIFNλR1) specific for the IFNλR1 gene (FIG. 5A).

IFNλ4 stimulation has been reported to lead assembly of the ISGF3 transcription factor complex consisting of phospho-STAT1, phospho-STAT2 and IRF9, and induce the expression of ISG15, which is important for antiviral activity (27, 28). As can be seen from FIGS. 5B and 5C, M1, M3 and M7 IFNλ4 variants induced the expression of ISG15 and inhibited HCV replication in HCV-infected Huh-7.5 cells. Interestingly, the M1, M3 and M7 IFNλ4 variants exhibited much higher ISG induction and antiviral activity than eIFNλ4.

Prolonged exposure to IFN2 proteins induces the production of non-phosphorylated ISGF3 (U-ISGF3) consisting of STAT1, STAT2 and IRF9 without tyrosine phosphorylation, whereas expression of phosphorylated ISGF3 is reduced (PLoS One 2012;7:e43960). As a result, upregulation of U-ISGF3-specific gene sets such as Mx1 is maintained for a long time. In order to determine whether or not long-term treatment with the M1, M3 and M7 IFNλ4 variants caused similar functions, the protein levels of STAT1, STAT2 and IRF9 were found to be equally upregulated by all IFNλ4 by measuring the protein levels of the U-ISGF3 component (FIG. 5D). In addition, it was found that the IFNλ4 variant has much better ability to maintain upregulation of Mx1 than eIFNλ4, but IFNλ1, IFNλ2 and IFNλ3 maintained stronger upregulation of Mx1 expression compared to the IFNλ4 variant.

Previously, eIFNλ4 has been reported to induce expression of negative regulators of IFN signaling, such as SOCS1 and USP18 (Sci. Rep. 2017;7:3821; Journal of Immunology 2017;199:3808-20). In order to evaluate the effect of glycosylation on the expression of IFN signaling negative regulators, the expression levels of SOCS1 and USP18 when treating Huh7 cell lines with M1, M3 and M7 variants were investigated. Treatment with IFNλ1, IFNλ2 and IFNλ3 significantly increased the level of USP18, whereas treatment with eIFNλ4 slightly increased the level of USP18. However, IFNλ4 variants (M1, M3 and M7) exhibited activity similar to IFNλ1, IFNλ2 and IFNλ3 (FIG. 5F). Protein expression of SOCS1 was not significantly increased by treatment with any type of IFNλ (FIG. 5F). However, treatment with IFNλs slightly upregulated the expression of SOCS1 mRNA without differences between the IFNλs types (FIG. 5G).

The results of Example above show that the structure-based approach for novel glycosylation selection according to the present invention can maintain the biological activity of IFNλ4, and in particular, the IFNλ4 variant of the present invention expressed from HEK293 exhibits remarkably better activity than eIFNλ4. It can be seen from the results of IFNλ4 variants that the major action residues of interferon lambda can be conserved based on the design characteristics, and thus can be extended to the entire type III interferons (IFNλ1 to IFNλ4) acting with the same receptor. A number of clinical trials on SARS-CoV-2 infection (COVID-19), which arises as a recent global pandemic, through treatment with interferon lambda have been reported (clinical number: NCT04388709; NCT04354259), and the effects thereof on MERS have been reported in the literature (Antiviral Res. 2020 August; 180: 104860). It is obvious in consideration of these circumstances that the interferon lambda variant with remarkably excellent productivity and antiviral properties according to the present invention and the method of producing the same can be more practical and effective for the prevention and treatment of COVID-19.

### INDUSTRIAL APPLICABILITY

The novel interferon lambda variant and the method of producing the same according to the present invention exhibit remarkably improved production and yield in mammalian cell lines using structural information-based glycoengineering, even through conventional purification protocols, and exhibit significantly improved therapeutic properties such as stability, half-life, and fraction of functional proteins during treatment compared to wild-type interferon lambda. In addition, the novel interferon lambda variant and the method of producing the same according to the present invention have higher antiviral activity and interferon-stimulated gene (ISG)-inducing activity than wild-type interferon lambda, and thus are useful for the prevention and treatment of immune-related diseases such as cancer and autoimmune diseases as well as various viral infections such as infection with the SARS-CoV-2 (COVID-19).

Although specific configurations of the present invention have been described in detail, those skilled in the art will appreciate that this description is provided to set forth preferred embodiments for illustrative purposes and should not be construed as limiting the scope of the present invention. Therefore, the substantial scope of the present invention is defined by the accompanying claims and equivalents thereto.

1. An interferon lambda (IFNλ) variants comprising a mutation at at least one site that satisfies at least one of the following criteria:
(i) a site is positioned outside an interferon lambda receptor-binding region;
(ii) a varied amino acid residue is exposed to a surface of interferon lambda; and
(iii) a consensus sequence enabling glycosylation is achieved through a single point amino acid mutation,
wherein the consensus sequence enabling glycosylation is N-X-(S or T), in which X is an amino acid other than proline.

2. The interferon lambda (IFNλ) variants according to claim 1, wherein the interferon lambda receptor in the criterion (i) is IL10Rβ or IFNλR1.

3. The interferon lambda (IFNλ) variants according to claim 1, wherein at least one site of the interferon lambda is glycosylated by the mutation.

4. The interferon lambda (IFNλ) variants according to claim 3, wherein the glycosylation is N-glycosylation.

5. The interferon lambda (IFNλ) variants according to claim 1, wherein the interferon lambda variants has a reduced net charge than a wild-type interferon lambda.

6. The interferon lambda (IFNλ) variants according to claim 1, wherein the interferon lambda variants has a longer in-vivo half-life than a wild-type interferon lambda.

7. The interferon lambda (IFNλ) variants according to claim 1, wherein the interferon lambda variants has a reduced hydrophobic interaction between interferon lambda molecules than a wild-type interferon lambda.

8. The interferon lambda (IFNλ) variants according to claim 1, wherein the interferon lambda is interferon lambda 4 (IFNλ4).

9. The interferon lambda (IFNλ) variants according to claim 8, wherein the mutation is substitution of an amino acid at at least one position of L28, A54, P73, H97, K154 and A173 in SEQ ID NO: 20 with asparagine.

10. The interferon lambda (IFNλ) variants according to claim 8, wherein the mutation is substitution of an amino acid at at least one position of L28N and P73N in SEQ ID NO: 20 with asparagine.

11. A gene encoding the interferon lambda variants according to claim 1.

12. A recombinant vector comprising the gene according to claim 11.

13. A recombinant host cell introduced with the gene according to claim 11 or a recombinant vector comprising said gene.

14. A composition for immunomodulation comprising the interferon lambda variants according to claim 1.

15. A pharmaceutical composition for preventing and treating viral infection comprising the interferon lambda variants according to claim 1.

16. A pharmaceutical composition for preventing and treating cancer, tumors, transplant rejection, chronic renal failure, cirrhosis, diabetes or hyperglycemia comprising the interferon lambda variants according to claim 1.

17. A method of producing an interferon lambda variants comprising:
culturing the recombinant cell according to claim 13 to express an interferon lambda variants comprising a mutation at at least one site of interferon lambda that satisfies at least one of the following criteria:
(i) a site is positioned outside an interferon lambda receptor-binding region;
(ii) a varied amino acid residue is exposed to a surface of interferon lambda; and
(iii) a consensus sequence enabling glycosylation is achieved through a single point amino acid mutation,
wherein the consensus sequence enabling glycosylation is N-X-(S or T), in which X is an amino acid other than proline; and
collecting the expressed interferon lambda variant.

18. A method for immunomodulation comprising administering the interferon lambda variants according to claim 1 to a subject.

19. A method for preventing and treating viral infection comprising administering the interferon lambda variants according to claim 1 to a subject.

20. A method for preventing and treating cancer, tumors, transplant rejection, chronic renal failure, cirrhosis, diabetes or hyperglycemia comprising administering the interferon lambda variants according to claim 1 to a subject.
